(19) United States
(12) Patent Application Publication
(10) Pub. No.: US 2016/0154928 A1
(43) Pub. Date: Jun. 2, 2016

(54) SYSTEMS, METHODS, AND ENVIRONMENT FOR AUTOMATED REVIEW OF GENOMIC DATA TO IDENTIFY DOWNREGULATED AND/OR UPREGULATED GENE EXPRESSION INDICATIVE OF A DISEASE OR CONDITION

(71) Applicant: IMMUNEERING CORPORATION, Cambrige, MA (US)

(72) Inventors: Benjamin Zeskind, Cambridge, MA (US); Kevin D. Fowler, Kevin, MA (US); Jason M. Funt, Cambridge, MA (US); Fadi Towfic, Cambridge, MA (US); Sarah E. Kolitz, Cambridge, MA (US)

(21) Appl. No.: 14/904,279

(22) PCT Filed: Jul. 11, 2014
(86) PCT No.: PCT/US14/46278

§ 371 Date: Jan. 11, 2016

Related U.S. Application Data
(60) Provisional application No. 61/845,940, filed on Jul. 12, 2013.
(60) Provisional application No. 61/879,878, filed on Sep. 19, 2013.

(51) Int. Cl. G06F 19/18; G06F 19/22; C07K 16/18; C12Q 1/68; A61K 31/13; G06F 19/20; G06F 19/26

(52) U.S. Cl. CPC G06F 19/18; G06F 19/20; G06F 19/22; G06F 19/26; C12Q 1/6883; A61K 31/13; C07K 16/18; C12Q 2600/156; C12Q 2600/158

(57) ABSTRACT

The disclosure relates to systems and methods for automated review of genomic data to identify genetic features indicative of a particular disease or condition. The system accesses genomic data of a first cohort of individuals and identifies one or more genes each of which is differentially expressed by individuals in a group having the disease or condition compared with a control group. The system accesses single-nucleotide polymorphism (SNP) data of a second cohort of individuals different from the first cohort and identifies SNPs associated with the disease or condition. The system determines an intersection between the set of identified genes and the SNPs associated with the disease or condition to identify one or more genes that are downregulated due to the disease or condition. Related treatment methods are also included.

### RELATED APPLICATIONS

The present application claims priority to and the benefit of U.S. Provisional Patent Application Ser. No. 61/845,940, filed Jul. 12, 2013, and U.S. Provisional Patent Application Ser. No. 61/879,878, filed Sep. 19, 2013, the content of each of which is hereby incorporated by reference herein in its entirety.

### BACKGROUND

Despite extensive efforts over many decades, the exact genetic and environmental causes of Alzheimer's Disease (AD) remain elusive. Staggering numbers of patients await effective medicines, and the field urgently needs new ideas and new targets. With respect to AD, as well as many other disease and conditions, there is a need for improved methods for mining existing genome data sets to derive information that may be used to detect and treat various diseases and conditions.

### SUMMARY

The present disclosure relates to systems, methods, and an environment for automated or semi-automated review of genomic data to identify genetic features indicative of a particular disease or condition. In some embodiments, a genetic feature is indicative of a particular disease or condition if its presence, level, form, or type shows a statistically significant correlation with incidence, presence, extent, or character of a disease, disorder, condition, state, or symptom or phenotype thereof. In some embodiments, the relevant presence, level, form or type of a genetic feature is in a particular location (e.g., cell type, tissue, or set thereof) and/or at a particular time (e.g., period of development, etc.). In some particular embodiments, the relevant presence, level, form, or type of a genetic feature is or comprises its presence, level form, or type in the brain.

In some aspects, the present disclosure provides systems for defining, identifying, and/or characterizing an appropriate correlation. In some aspects, the present disclosure provides systems for detecting correlated features. The present disclosure particularly provides systems that analyze data from two or more different patient cohorts.

In some embodiments, categories of populations are divided by subsets of individuals having differential gene expressions of certain disease marker or markers. Individuals include patients, healthy or normal individuals, as well as those at risk of developing a disease or a condition. Patients include those who have been diagnosed with a disease or a condition and those who have a disease or a condition but have not been diagnosed. In some embodiments, a disease or a condition manifests itself as symptomatic or asymptomatic.

In some embodiments, such subsets involve a genotype of a marker (i.e., a marker gene). More, specifically, one subset represents a population of individuals having a “positive (+)” genotype, while a second subset represents a population of individuals having a “negative (−)” genotype. An individual with a positive genotype is generally referred to as a carrier of a particular allele. An individual with a negative genotype is generally referred to as a non-carrier of a particular allele.

In some embodiments, such subsets involve categorizing by the gender of individuals in a population. In some embodiments, a disease or a condition of interest exhibits gender-dependent features, such as differential pathogenesis, including differences in the onset, severity, duration, survival, and/or symptoms of a disease or condition.

The present disclosure takes into account that subsets of individuals may show differential responsiveness to a particular therapy, including types of drugs, effective dosage and other therapeutic regimens, side effects, and so on. In some embodiments, subsets of individuals show differential responsiveness to different combinations of drugs (e.g., combination therapy). As used herein, differential responsiveness refers to statistically significant variations observable within a population of individuals in response to a particular therapy.

The present disclosure provides systems for performing data analysis and/or for identification, detection, and/or characterization of genetic features indicative of a particular disease or condition. The present disclosure furthermore provides systems (e.g., methods, reagents, kits) for detecting such genetic features in populations suffering from, susceptible to, and/or receiving treatment for the disease or condition.

In some embodiments, a “genetic feature” is or comprises an expression level of a gene or gene product, a form of a gene or gene product (e.g., methylation state of a gene; capping or spliced condition of an RNA gene product, phosphorylation state of a protein gene product, etc.), an activity level with respect to at least one biological function or type of a gene or gene product, and/or a genetic marker (e.g., a single nucleotide polymorphism (“SNP”) or other sequence variation, copy number variation, heterogeneity, etc.), wherein the genetic feature is associated or correlated with a particular disease, disorder, condition, state, or symptom or phenotype thereof.

Those skilled in the art will appreciate that, once a particular genetic feature is identified as of interest as described herein for use in diagnosing or monitoring of individuals suffering from, susceptible to, and/or receiving treatment for a disorder or condition, subsequent detection and/or measurement of that genetic feature (and/or its location and/or timing) may be direct (i.e., through direct detection of the feature itself in one or more relevant location(s) and/or at one or more relevant time(s)) or by proxy (i.e., through detection of a marker correlated with, indicating or revealing the relevant genetic feature). Those skilled in the art are well aware of the enormous number of technologies (e.g., hybridization, sequencing, amplification of nucleic acids and/or binding [e.g., with antibodies, or other ligands], or activity assays for proteins), well established in the field as useful in the detection and/or measurement of genetic features and/or proxies therefore. The challenge in the industry, in many instances, is not how to detect or measure genetic features of interest once identified, but rather to know which genetic features, or combinations thereof should desirably be so detected in order to yield meaningful information. The present invention provides technologies that define sets of genetic features that, when detected and/or measured, can provide meaningful information. The present disclosure further provides analysis technologies that permit and achieve the extraction of such useful information (e.g., degree of risk that a patient will develop a particular disease, disorder, condition, state, or symptom thereof [e.g., within a particular time window], will respond or is responding to a particular therapeutic regimen, or will develop or is developing a particular side effect of therapy or symptom or type of the disease, disorder, or condition, etc.).

In one aspect, genomic data of a first cohort of individuals, including a case group having the disease or condition and a control group not exhibiting the disease or condition, is automatically reviewed to identify genes that are differentially expressed by the individuals in the case group as compared with the individuals of the control group. SNP (or other genetic marker) data of a second cohort of individuals, having partial or no overlap with the first cohort of individuals, is automatically reviewed to identify one or more markers (e.g., SNPs) associated with the disease or condition of the case group. The differentially expressed genes identified with reference to the first cohort of individuals are then analyzed in view of the markers identified with reference to the second cohort of individuals to determine an intersection of one or more genes which are downregulated and/or upregulated due to the disease or condition.

In some embodiments, the case group of second cohort is separated into subsets by demographic information and/or gene information. For example, the second cohort may be divided into subsets based at least in part upon sex, age, and/or other demographic information. In another example, the second cohort may be divided into subsets based at least in part upon polymorphic expression status, such as APOE status within an Alzheimer case group. In this manner, differentially expressed genes may be identified on a per-subset basis.

In another aspect, the present disclosure relates to systems and methods for calculating a propensity score representing a measure of preference of a particular genetic marker (e.g., SNP genotype) to case subsets versus control subsets of a given data set. Propensity score values, in some embodiments, are graphically illustrated to enable a user to quickly distinguish allelic variants that have strong indication to be associated with case or control classes or groups within a particular study, such as a genome wide association study (GWAS).

In another aspect, the present disclosure relates to systems and methods for searching large gene expression datasets containing un-normalized or partially-normalized data for samples matching a particular gene signature or expression profile. For example, a researcher may want to target a specific pathway for upregulation in an experiment. The researcher may search for the particular profile and limit the data output to samples in which the specific profile is upregulated. In some embodiments, a normalized enrichment score is calculated for each sample within each of the large datasets. The normalized enrichment score represents a measure of whether a given input gene set of two or more genes is upregulated, downregulated, or both in a given sample. The normalized enrichment score may then be converted to z-scores having a standard Gaussian distribution, thereby facilitating fast computation of p-values. The normalized enrichment score for a given sample, in some examples, can include one or more of a) a measure of significance of differential expression of probes annotated to a gene of interest against all other probes in the sample, b) a signal-to-noise ratio associated with the input genes in the sample compared to other genes in the sample, and c) a difference between the number of genes in the sample and the number of genes in the input gene set.

In another aspect, the present disclosure relates to a system for identifying one or more genes that are downregulated due to a disease or condition. In some embodiments, the disease or condition is Alzheimer's disease (AD). The system includes a processor and a memory having instructions stored thereon where the instructions, when executed by the processor, cause the processor to (a) access genomic data of a first cohort of individuals, where the first cohort includes a group of individuals having the disease or condition and a control group of individuals that do not have the disease or condition. The instructions, when executed by the processor, cause the processor to (b) identify, from the genomic data of at least a subset of (e.g., a subcategory, e.g., gender and/or gene marker status, e.g., APOE status) the first cohort, a set of one or more genes each of which is differentially expressed by individuals in the group having the disease or condition compared with the control group. The instructions, when executed by the processor, cause the processor to (c) access single-nucleotide polymorphism (SNP) data of a second cohort of individuals different from the first cohort (e.g., there may be some overlap between the first and second cohorts, or there may be no members in common between the first and second cohorts). The instructions, when executed by the processor, cause the processor to (d) identify, from the SNP data of at least a subset of (e.g., a subcategory, e.g., gender and/or gene marker status, e.g., APOE status) the second cohort, a plurality of SNPs associated with the disease or condition (e.g., using a subset-specific Genome-Wide Association Study, GWAS). The instruction, when executed by the processor, cause the processor to (e) determine an intersection between the set of one or more genes identified in (b) and the SNPs associated with the disease or condition identified in (d) to identify one or more genes that are downregulated due to the disease or condition.

In some embodiments, the instructions, when executed by the processor, cause the processor to (f) access a drug database and (g) identify one or more drug candidates for restoring expression of at least one of the one or more downregulated genes identified in (e).

In another aspect, the present disclosure relates to a system for visualizing location and/or significance of a set of identified single-nucleotide polymorphisms (SNPs) in relation to one or more identified gene via propensity plotting (e.g., for determining an intersection between the one or more identified genes and the set of SNPs to identify one or more genes associated with a disease or condition that is/are downregulated due to the disease or condition). In some embodiments, the disease or condition is Alzheimer's disease (AD). The system includes a processor and a memory having instructions stored thereon, where the instructions, when executed by the processor, cause the processor to determine, for each of one or more SNPs identified in a Genome-Wide Association Study (GWAS) of a dataset, a propensity score for each of one or more allelic states of the SNP. The propensity score for a given allelic state provides a measure of prevalence of the allelic state of the SNP in a case subset versus a control subset of the dataset, where the case subset corresponds to subjects with a given disease or condition and the control subset corresponds to subjects who do not have the disease or condition. The instructions, when executed by the processor, cause the processor to display, for each of the SNPs identified in the GWAS of the dataset, a graphical representation of the propensity score for each of the one or more allelic states of the SNP, thereby enabling a user to distinguish allelic states having strong association with either the case subset or the control subset of the dataset.

In some embodiments, the graphical representation includes an x-y plot, with each of one or more allelic states of a given SNP represented by a discrete location along either the x or y axis, and a value of the propensity score (e.g., log 2 value) represented graphically (e.g., via bar height) along the other axis.

In another aspect, the present disclosure describes a system for performing a search of one or more large datasets containing gene expression data (e.g., the NIH GEO datasets and/or CMAP/Connectivity Map datasets), at least a portion of which is not normalized (e.g., dataset includes subsets of data from different sources, measured by different instruments, etc., where at least some of the subsets are not normalized with respect to each other), to identify samples in the one or more large datasets having an input gene set that is significantly upregulated only, downregulated only, or either up OR downregulated. The system includes a processor and a memory having instructions stored thereon, where the instructions, when executed by the processor, cause the processor to determine a normalized enrichment score for each of a plurality of samples in the one or more large datasets, where the normalized enrichment score for a given sample is a measure of whether a given input gene set including one or more genes are upregulated, downregulated, or both in the given sample.

In some embodiments, the normalized enrichment score for a given sample includes one or more of: (i) a measure of significance of differential expression of probes annotated to a gene of interest against all other probes in the sample; (ii) a signal-to-noise ratio associated with the input genes in the sample compared to other genes in the sample; and (iii) a difference between the number of genes in the sample and the number of genes in the input gene set.

The instructions, when executed by the processor, cause the processor to convert the normalized enrichment score for one or more samples to z-scores having a standard Gaussian distribution (thereby facilitating fast computation of p-values). The instructions, when executed by the processor, cause the processor to identify a subset of the samples in the large datasets in which the given input gene set is upregulated, downregulated, or both (e.g., identify a subset of samples in which a specific signature/expression profile corresponding to the input gene set occurs).

In some embodiments, the instructions, when executed by the processor, cause the processor to identify conditions and/or treatments that upregulate (or downregulate) a given pathway.

In some embodiments, the instructions, when executed by the processor, cause the processor to identify one or more other conditions and/or diseases whose expression profiles are similar to that of a disease of interest (e.g., where the disease of interest is a disease or condition in which it is known that the input gene set is significantly upregulated, downregulated, or either up OR downregulated). In some embodiments, the disease or condition is Alzheimer's disease (AD).

In some embodiments, the instructions, when executed by the processor, cause the processor to use the identified other conditions and/or diseases whose expression profiles are similar to that of the disease of interest to identify one or more pathways common between the identified other conditions and/or diseases and the disease of interest (e.g., based on CMAP/Connectivity Map dataset).

In some embodiments, the instructions, when executed by the processor, cause the processor to use the identified other conditions and/or diseases whose expression profiles are similar to that of the disease of interest to identify one or more known treatments for the other conditions and/or diseases (e.g., which can be used as a treatment for the disease of interest) (e.g., based on CMAP/Connectivity Map dataset).

In another aspect, the present disclosure describes a method for identifying one or more genes that are downregulated due to a disease or condition. In some embodiments, the disease or condition is Alzheimer's disease (AD). The method includes (a) identifying, by a processor of a computing device, a set of one or more genes each of which is differentially expressed by individuals in a group having the disease or condition compared with individuals in a control group that do not have the disease or condition. The identifying is based on data corresponding to a first cohort of individuals. The method includes (b) accessing, by the processor, single-nucleotide polymorphism (SNP) data of a second cohort of individuals different from the first cohort (e.g., there may be some overlap between the first and second cohort, or there may be no members in common between the first and second cohorts) and identifying, by the processor, SNPs associated with the disease or condition (e.g., using a subset-specific Genome-Wide Association Study, GWAS). The method includes (c) determining, by the processor, an intersection between the set of one or more genes identified in step (a) and the SNPs associated with the disease or condition identified in step (b) to identify one or more genes that are downregulated due to the disease or condition.

In some embodiments, the method further includes (d) accessing, by the processor, a drug database and identifying, by the processor, one or more drug candidates for restoring expression of at least one of the one or more downregulated genes.

In some embodiments, the downregulated genes identified in step (c) is/are indicative of an upstream signal (e.g., causative of the disease or condition) rather than a downstream signal resulting from disease pathology.

In another aspect, the present disclosure describes a method for performing a search of one or more large datasets containing gene expression data (e.g., the NIH GEO datasets and/or CMAP/Connectivity Map datasets), at least a portion of which is not normalized (e.g., dataset includes subsets of data from different sources, measured by different instruments, etc., where at least some of the subsets are not normalized with respect to each other), to identify samples in the one or more large datasets having an input gene set that is significantly upregulated only, downregulated only, or either up OR downregulated. The method includes determining, by a processor of a computer, a normalized enrichment score for each of a plurality of samples in the large datasets, where the normalized enrichment score for a given sample is a measure of whether a given input gene set including one or more genes are upregulated, downregulated, or both in the given sample.

In some embodiments, the normalized enrichment score for a given sample includes one or more of: (i) a measure of significance of differential expression of probes annotated to a gene of interest against all other probes in the sample; (ii) a signal-to-noise ratio associated with the input genes in the sample compared to other genes in the sample; and (iii) a difference between the number of genes in the sample and the number of genes in the input gene set.

The method includes converting, by the processor, the normalized enrichment score for one or more samples to z-scores having a standard Gaussian distribution (thereby facilitating fast computation of p-values). The method includes identifying, by the processor, a subset of the plurality of samples in the one or more large datasets in which the given input gene set that is upregulated, downregulated, or both (e.g., identify a subset of samples in which a specific signature/expression profile corresponding to the input gene set occurs).

In some embodiments, the method includes identifying, by the processor, conditions and/or treatments that upregulate (or downregulate) a given pathway.

In some embodiments, the method includes identifying, by the processor, one or more other conditions and/or diseases whose expression profiles are similar to that of a disease of interest (e.g., where the disease of interest is a disease or condition in which it is known that the input gene set is significantly upregulated, downregulated, or either up OR downregulated). In some embodiments, the disease or condition is Alzheimer's disease (AD).

In some embodiments, the method includes using (e.g., by the processor) the identified other conditions and/or diseases whose expression profiles are similar to that of the disease of interest to identify one or more pathways common between the identified other conditions and/or diseases and the disease of interest (e.g., based on CMAP/Connectivity Map dataset).

In some embodiments, the method includes using (e.g., by the processor) the identified other conditions and/or diseases whose expression profiles are similar to that of the disease of interest to identify one or more known treatments for the one or more other conditions and/or diseases (e.g., which can be used as a treatment for the disease of interest) (e.g., based on CMAP/Connectivity Map dataset).

In another aspect, the present disclosure describes a method that includes steps of: determining one or more of gender and ApoE4 status for a subject and detecting in samples from the subject a genetic feature. The genetic feature is indicative of NEUROD6 expression, activity, or combination thereof in the subject's brain (the “NEUROD6 feature”) as compared with an appropriate reference (the “NEUROD6 reference”), indicative of SNAP25 expression, activity, or combination thereof in the subject (the “SNAP25 feature”) as compared with an appropriate reference (the “SNAP25 reference”), or combinations thereof.

In certain embodiments, the step of detecting a genetic feature includes obtaining a sample from the subject and processing the sample by contacting it with reagents sufficient to hybridize with or amplify NEUROD6 nucleic acids in the sample, or to bind to or react with NEUROD6 protein such that the subject's brain level of NEUROD6 is determined. The sample does not include brain tissue, in some embodiments, and the subject's brain level of NEUROD6 is determined by proxy.

In other embodiments, the step of detecting a genetic feature includes obtaining a sample from the subject and processing the sample by contacting it with reagents sufficient to hybridize with or amplify SNAP25 nucleic acids in the sample, or to bind to or react with SNAP25 protein. The sample may not include brain tissue, and the subject's brain level of SNAP25 is determined by proxy.

In some embodiments, the step of determining ApoE4 status in a subject includes obtaining a sample from the subject and processing the sample by contacting it with reagents sufficient to hybridize with or amplify ApoE4 nucleic acids in the sample, or to bind to or react with ApoE4 protein.

In some embodiments, the method includes a step of administering Alzheimer's therapy, including one or more agents, to the subject if the subject is either: i) ApoE4+ female and has a NEUROD6 feature indicating a level, expression, activity, or function of NEUROD6 in the subject's brain that is significantly lower than that of a normal NEUROD6 reference or ii) ApoE4+ male and has a SNAP25 feature indicating a level, expression, activity, or function of SNAP25 expression in the subject's brain that is significantly lower than that of a normal SNAP25 reference.

In certain embodiments, the step of administering includes administering an agent whose administration correlates with increased NEUROD6 brain level, expression, function, or activity. In some embodiments, the NEUROD6 feature is or includes a SNP. In certain embodiments, the step of detecting a genetic feature includes obtaining a sample from the subject and processing the sample by contacting it with reagents sufficient to hybridize with or amplify the SNP.

In some embodiments, the NEUROD6 reference is or includes a NEUROD6 brain level, expression, function, or activity in normal females.

In other embodiments, the step of administering includes administering an agent whose administration correlates with increased SNAP25 brain level, expression, function, or activity. In some embodiments, the agent is selected from, or includes portions of, the following: valproic acid, guanabenz, karakoline, tetracycline, diloxanide, metoprolol, yohimbic acid, azapropazone, proguanil, and combinations thereof.

In some embodiments, the SNAP25 feature is or includes a SNP. The step of detecting a genetic feature may include obtaining a sample from the subject and processing the sample by contacting it with reagents sufficient to hybridize with or amplify the SNP.

In some embodiments, the NEUROD6 reference or the SNAP25 reference is a level or range or expression, function, or activity observed in a population of normal individuals not suffering from or being treated for Alzheimer's Disease. In some embodiments, the NEUROD6 reference or the SNAP25 reference is a historical reference. In some embodiments, the NEUROD6 reference or the SNAP25 reference is a reference level, expression, function, or activity determined in a sample from the subject at an earlier time.

In certain embodiments, the agent is selected from, or includes portions of: sodium phenylbutyrate, arachidonic acid, 2-deoxy-D-glucose, fasudil, nordihydroguaiaretic acid, monastrol, tacrolimus, quercetin, sulindac, troglitazone, staurosporine, troglitazone, thalidomide, CP-944629, mercaptopurine, haloperidol, exisulind, sirolimus, tanespimycin, suramin sodium, genistein, erastin, clofibrate, LY-294002, tanespimycin, LY-294002, prednisolone, fulvestrant, meteneprost, monorden, tretinoin, nifedipine, sulindac, ulfide, wortmannin, MK-886, PF-01378883-00, monorden, iloprost, or combinations thereof.

In other embodiments, the agent is or includes a cholinesterase inhibitor. In some embodiments, the agent is or includes donepezil, rivastigmine, or galantamine.

In another embodiment, the agent is or includes a glutamate regulator. In some embodiments, the agent is or includes memantine.

In another embodiment, the agent is or includes an antidepressant, an anxiolytic, or an antipsychotic. In some embodiments, the antidepressant includes citalopram, fluoxetine, paroxetine, sertraline, or combinations thereof the anxiolytic includes lorazepam, oxazepam, or combinations thereof and the antipsychotic includes ariprazole, baloperidol, olanzapine, or combinations thereof.

In another embodiment, the agent is or includes a beta secretase inhibitor, a gamma secretase inhibitor, or combinations thereof.

In another embodiment, the agent is or includes an antibody agent that binds specifically to amyloid beta or tau. In some embodiments, the antibody agent is an intact antibody, an antigen-binding fragment thereof, or combination thereof.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1 is a flow diagram of an example flow for automated review of genomic data to identify downregulated and/or upregulated gene expression indicative of a disease or condition;

FIG. 2 is a block diagram of a system for automated review of genomic data to identify downregulated and/or upregulated gene expression indicative of a disease or condition;

FIG. 3 is a flow chart of an example method for identification of drug candidates for therapy of patients having a particular disease or condition based upon automated review of genomic data to identify downregulated and/or upregulated gene expression;

FIG. 4 is a flow chart of an example method for determining and presenting propensity scores related to single-nucleotide polymorphisms;

FIG. 5 is an example propensity score display related to a given SNP;

FIG. 6 is a flow chart of an example method for mining large datasets of un-normalized or partially normalized gene expression data for samples exhibiting a particular signature or expression profile;

FIG. 7 is a block diagram of an example network environment for automated review of genomic data to identify downregulated and/or upregulated gene expression indicative of a disease or condition;

FIG. 8 is a block diagram of a computing device and a mobile computing device;

FIG. 9 is an example Venn diagram that illustrates intersections of significantly downregulated genes across a given set of expression datasets;

FIGS. 10A-10E are example box plots of NEUROD6 expression, activity, or combination that illustrate the consistent downregulation of identified genes across a given set of expression datasets;

FIGS. 11A-11D are example plots that illustrate SNPs in the region of NEUROD6 that are found to be associated with Alzheimer's disease (AD) in certain population groups;

FIGS. 12A-12D are example propensity plots that show propensity scores for disease risk or protection of NEUROD6 SNPs in certain population groups determined from a given set of expression datasets;

FIGS. 13A-13E are example box plots of SNAP25 expression, activity, or combination that illustrate the consistent downregulation of the identified genes across a given set of expression datasets;

FIGS. 14A-14D are example plots that illustrate SNPs in the region of SNAP25 in APOE4+ that are found to be associated with Alzheimer's disease (AD) in certain population groups;

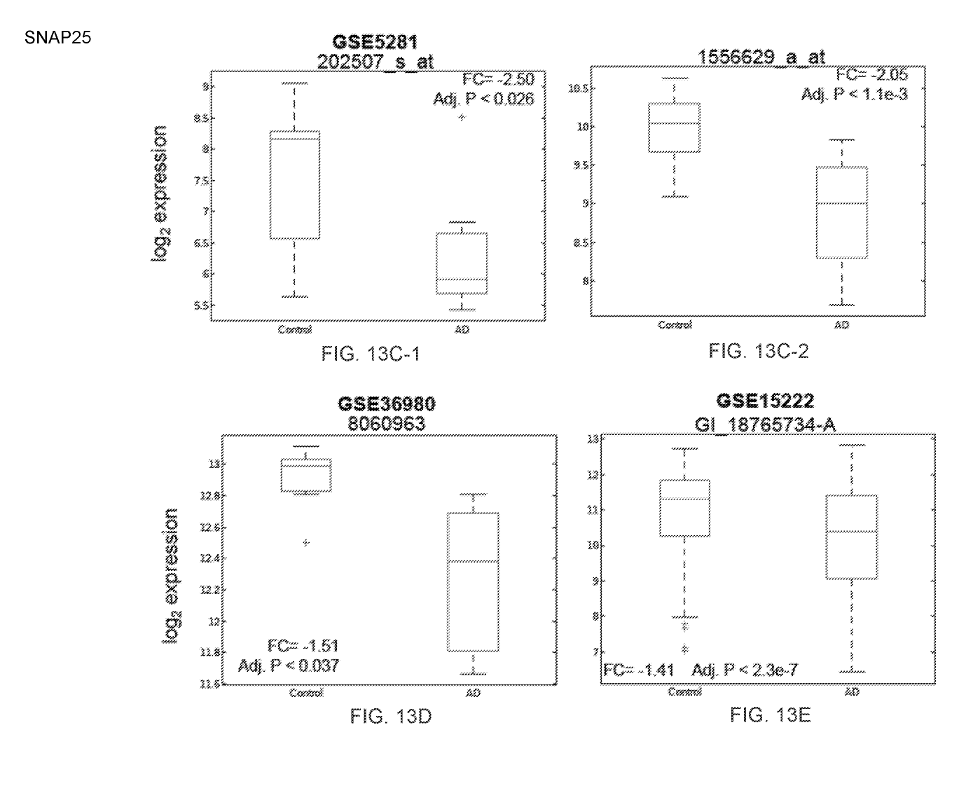

FIGS. 15A-15B are example propensity plots that show propensity scores for disease risk or protection of SNAP25 SNPs in certain population groups determined from a given set of expression datasets;

FIG. 16 is an example plot of a specificity heat map for NEUROD6 in certain brain tissues;

FIG. 17 is a plot that illustrates a distribution of samples with high NEUROD6 expression among a male and female population within a dataset of healthy candidates;

FIG. 18 is an example box plot that illustrates NEUROD6 expression in male and female populations across a dataset of healthy candidates; and

FIGS. 19A-19D are example box plots that illustrate NEUROD6 expressions in male and female populations across a number of tissue types.

Various features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

Amino acid: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

Animal: As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal. In some embodiments, an antibody has constant region sequences that are characteristic of mouse, rabbit, primate, or human antibodies. In some embodiments, an antibody sequence elements are humanized, primatized, chimeric, etc, as is known in the art.

Antibody agent: The term “antibody agent”, as used herein, refers to agents that include one or more antibody structural features. In many embodiments, such agents show specific binding characteristics also found in antibodies. In some embodiments, antibody agents are or comprise intact antibodies, or fragments thereof. In some embodiments, the term can refer to bi- or other multi-specific (e.g., zybodies, etc) antibodies, Small Modular ImmunoPharmaceuticals (“SMIPs™”), single chain antibodies, cameloid antibodies, and/or antibody fragments. In some embodiments, an antibody agent may lack a covalent modification (e.g., attachment of a glycan) that an antibody would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc].

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc.

Antigen: The term “antigen”, as used herein, refers to an agent that elicits an immune response; and/or (ii) an agent that binds to a T cell receptor (e.g., when presented by an MEW molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity, such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element. In certain embodiments, particular characteristic sequence elements may be or include “motifs”.

Combination therapy: As used herein, the term “combination therapy” refers to those situations in which a subject is simultaneously exposed to two or more therapeutic agents. In some embodiments, such agents are administered simultaneously; in some embodiments, such agents are administered sequentially; in some embodiments, such agents are administered in overlapping regimens.

Comparable: The term “comparable”, as used herein, refers to two or more agents, entities, situations, sets of conditions, etc that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc to be considered comparable.

Corresponding to: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form: As used herein, the term “dosage form” or “dosage” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is correlated with a desired or beneficial outcome when administered across a relevant population (i.e., is a therapeutic dosing regimen).

Encapsulated: The term “encapsulated” is used herein to refer to substances that are completely surrounded by another material.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Functional: As used herein, the term “functional” is used to refer to a form or fragment of an entity that exhibits a particular property and/or activity.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Human: In some embodiments, a human is an embryo, a fetus, an infant, a child, a teenager, an adult, or a senior citizen.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. In some embodiments, isolation involves or requires disruption of covalent bonds (e.g., to isolate a polypeptide domain from a longer polypeptide and/or to isolate a nucleotide sequence element from a longer oligonucleotide or nucleic acid).

Modulator: The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Nanoparticle membrane: As used herein, the term “nanoparticle membrane” refers to the boundary or interface between a nanoparticle outer surface and a surrounding environment. In some embodiments, the nanoparticle membrane is a polymer membrane having an outer surface and bounding lumen.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, or more residues long.

Patient: As used herein, the term “patient” refers to a human or any non-human animal (e.g., mouse, rat, rabbit, dog, cat, cattle, swine, sheep, horse or primate) to whom therapy is administered. In many embodiments, a patient is a human being. In some embodiments, a patient is a human presenting to a medical provider for diagnosis or treatment of a disease, disorder or condition. In some embodiments, a patient displays one or more symptoms or characteristics of a disease, disorder or condition. In some embodiments, a patient does not display any symptom or characteristic of a disease, disorder, or condition. In some embodiments, a patient is someone with one or more features characteristic of susceptibility to or risk of a disease, disorder, or condition.

Pharmaceutically acceptable: The term “pharmaceutically acceptable” as used herein, refers to agents that, within the scope of sound medical judgment, are suitable for use in contact with tissues of human beings and/or animals without excessive toxicity, irritation, allergic response, or other problem or complication, commensurate with a reasonable benefit/risk ratio.

Polypeptide: The term “polypeptide”, as used herein, generally has its art-recognized meaning of a polymer of at least three amino acids, linked to one another by peptide bonds. In some embodiments, the term is used to refer to specific functional classes of polypeptides, such as, for example, autoantigen polypeptides, nicotinic acetylcholine receptor polypeptides, alloantigen polypeptides, etc. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15, or more contiguous amino acids. In some embodiments, a useful polypeptide as described herein may comprise or consist of a fragment of a parent polypeptide. In some embodiments, a useful polypeptide as described herein may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: The term “reference” is often used herein to describe a standard or control agent or value against which an agent or value of interest is compared. In some embodiments, a reference agent is tested and/or a reference value is determined substantially simultaneously with the testing or determination of the agent or value of interest. In some embodiments, a reference agent or value is a historical reference, optionally embodied in a tangible medium. Typically, as would be understood by those skilled in the art, a reference agent or value is determined or characterized under conditions comparable to those utilized to determine or characterize the agent or value of interest.

Refractory: As used herein, the term “refractory” refers to any subject that does not respond with an expected clinical efficacy following the administration of provided compositions as normally observed by practicing medical personnel.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Stable: The term “stable,” when applied to compositions herein, means that the compositions maintain one or more aspects of their physical structure over a period of time. In some embodiments, a stable provided composition is one for which a biologically relevant activity is maintained for a period of time. In some embodiments, the period of time is at least about one hour; in some embodiments the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc.

Subject: As used herein, the term “subject” refers to a human or any non-human animal (e.g., mouse, rat, rabbit, dog, cat, cattle, swine, sheep, horse or primate), or in some embodiments plant.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits or has exhibited one or more symptoms or characteristics of the disease, disorder, or condition.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc.)

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic agent: As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Therapeutic regimen: A “therapeutic regimen”, as that term is used herein, refers to a dosing regimen whose administration across a relevant population is correlated with a desired or beneficial therapeutic outcome.

Treatment: As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

Headers are used herein to aid the reader and are not meant to limit the interpretation of the subject matter described.

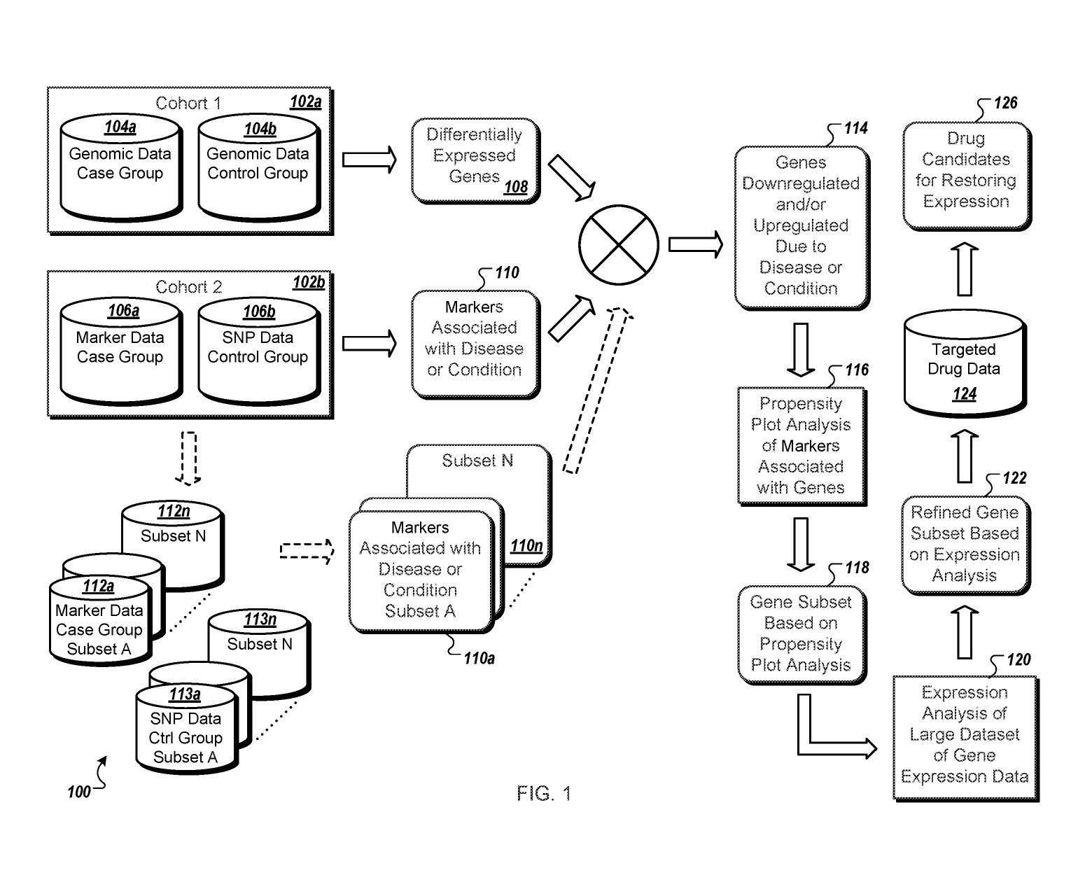

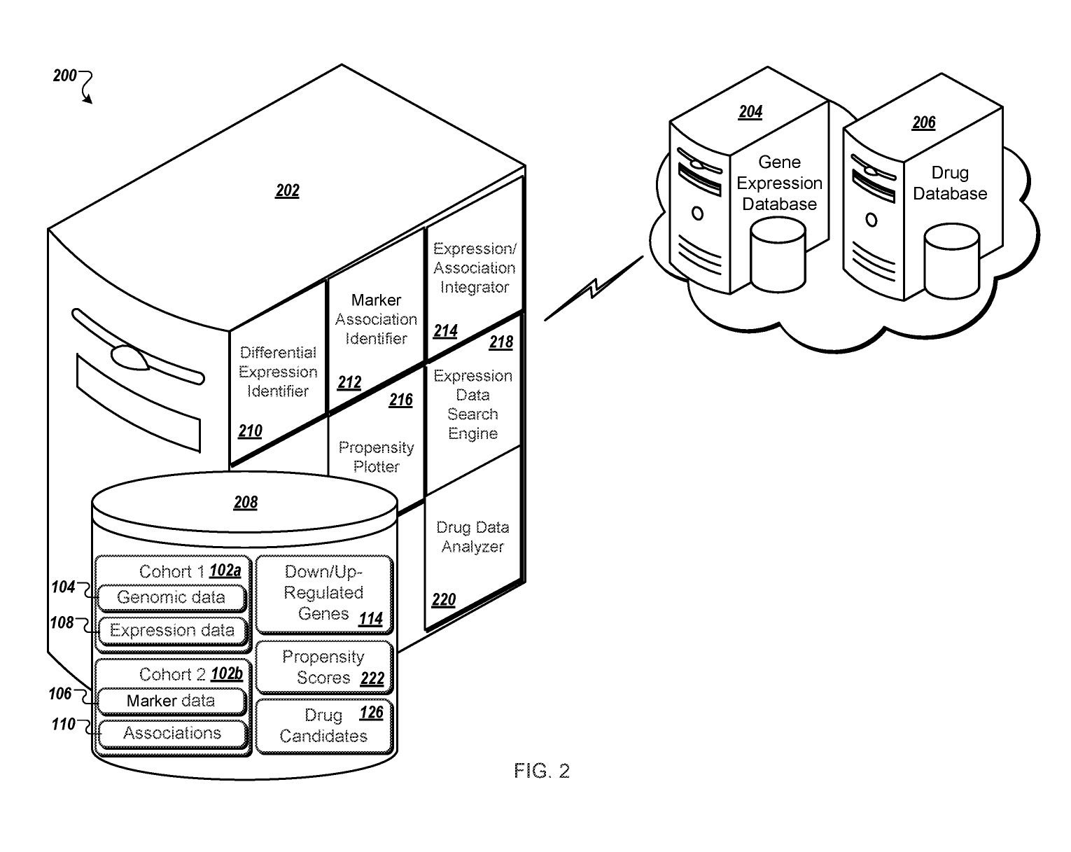

FIG. 1 is a flow diagram of an example flow 100 for automated review of genomic data to identify downregulated and/or upregulated gene expression indicative of a disease or condition. The flow 100, for example, may be used to identify candidate therapies for individuals or subsets of individuals with a particular disease or condition, such as Alzheimer's. At least a portion of the data collection, review, and analysis operations described in relation to the flow 100, in some implementations, is performed by a server 202 as illustrated in FIG. 2.

In some implementations, the flow 100 begins with accessing genomic data 104 related to a first cohort of individuals 102a and marker data 106 related to a second cohort of individuals 102b. The first cohort 102a may or may not have partial overlap with the second cohort 102b. For example, the genomic data 104 may or may not be accessed from a same resource as the marker data 106. The marker data 106, for example, may include Copy Number Alteration (CNA) or Copy Number Variation (CNV) data obtained through virtual karyotyping with SNP arrays, such as the Affymetrix Genome-Wide Human SNP 6.0 array by Affymetrix of Santa Clara, Calif. The genomic data 104, in some examples, may include data obtained as biological sequencing output from a next generation medical sequencer (e.g., paired-end sequencing, high throughput sequencing, etc.) or from other cytogenetic techniques, such as fluorescent in situ hybridization (FISH), comparative genomic hybridization (CGH), or array comparative genomic hybridization (ACGH). The genomic data 104 and/or the marker data 106 may be accessed from a public repository, such as, in some examples, the Alzheimer's Disease Neuroimaging Initiative (ADNI) database of UCLA, or the Gene Expression Omnibus (GEO) database. For example, turning to FIG. 2, the server 202 may access at least a portion of the genomic data 104 from one or more gene expression databases 204 via a network (e.g., wide area network, local area network, Internet, Intranet, etc.). The genomic data 104, once collected, may be stored in a storage medium 208, included in the server 202 or accessible to the server 202 via a wired or wireless connection. The storage medium 208 may include one or more storage devices accessible via a wireless network connection, such as a cloud storage region.

In some implementations, prior to analysis, the genomic data 104 and/or marker data 106 is filtered to select candidate samples for analysis. For example, to improve quality of the dataset, outlier samples may be removed based upon a number of criteria. Example criteria include detection scores, transcript prevalence (e.g., detected in a threshold minimum number of samples), and metadata filtering (e.g., disease stage, disease severity, presence or absence of secondary conditions, receipt [or lack or extent thereof] of particular therapy, and/or other data related to the patient). Additionally, the data may undergo preprocessing such as normalization, evaluation (in the case of raw data sets), and reformatting.

In some implementations, the genomic data 104 is analyzed to identify one or more differentially expressed genes 108. The case group genomic data 104a can be compared to the control group genomic data 104b using a number of analysis tools. For example, Linear Models for Microarray Data (LIMMA) may be used to determine probes significantly different between the case group genomic data 104a and the control group genomic data 104b. Significant difference, for example, may be associated with a multiple hypothesis corrected p-value less than 0.05. Genomic data analysis can be performed by a differential expression identifier module 210 (e.g., software algorithm, program, portion of a software tool suite, etc.) executed by the server 202, as illustrated in relation to FIG. 2.

In some implementations, the differentially expressed genes 108 are filtered based upon one or more criteria. For example, the downregulated probes of the differentially expressed genes 108 may be filtered according to a first threshold (e.g., marginal) average presence/absence call for all samples within the genomic data control group 104b, while the upregulated probes of the differentially expressed genes 108 may be filtered according to a second threshold (e.g., marginal) average presence/absence call within the genomic data control group 104b.

In some implementations, prior to cross-referencing the differentially expressed genes 108 with markers 110, the differentially expressed genes 108 are analyzed to identify tissue-specific expression. For example, in research of differentially expressed genes in Alzheimer's patients, the differentially expressed genes 108 may be sorted as being brain-associated or not. In one example, the probes of the differentially-expressed genes 108 are mapped to tissue specific arrays available via BioGPS.

In some implementations, the marker data 106a of the second cohort 102b is analyzed to identify markers associated with the disease or condition of the marker data case group 106a. For example, a Genome Wide Association Study (GWAS) may be conducted on the case group marker data 106a and the control group marker data 106b to identify the markers 110. In some implementations, a marker association identifier module 212 is executed by the server 202 to identify markers associated with the disease or condition. In a particular example, the PLINK software package may be utilized to conduct the GWAS on the marker data 106.

In some implementations, the marker data 106 is divided into two or more subsets 112, 113, for example, based upon demographic information (e.g., sex, age, etc.) and/or genomic information (e.g., polymorphic status of a gene of particular interest in relation to the disease or condition, etc.). In a particular example, the marker data 106 associated with an Alzheimer's study may be divided into case group subsets 112 and control group subsets 113 based upon both sex and APOE status.

In some implementations, the intersection between the differentially expressed genes 108 and the markers 110 is determined as genes 114 which are downregulated and/or upregulated due to the disease or condition. While differentially expressed genes are relevant to understanding disease biology, it can be difficult to determine which gene expressions are downstream responses to disease pathology and which gene expressions are more causative of the disease. To aid in identification of relevant upstream genes, the intersection of the differentially expressed genes 108 and the markers 110 is determined to identify genes 114 that are both differentially expressed in the disease or condition population and have polymorphisms significantly associated with the disease or condition. In some implementations, one or more markers 110 may be identified as being or mapped near each of at least a subset of the differentially expressed genes 108. For example, in a particular study of Alzheimer's data, three SNPs in and around NEUROD6 were identified as being significant to the APOE4+ subset 112 of the second cohort 102b.

In some implementations, an expression/association integrator module 214 is executed by the server 202 of FIG. 2 to determine the genes 114.

In some implementations, propensity analysis is conducted on the markers 110 associated with each of the genes 114. A propensity score provides a measure of preference of a particular SNP genotype to case 106a in view of control 106b datasets of the cohort 102b. A particular algorithm for propensity score calculation is as follows:

$$log 2 ( {PROPENSITY}_{rsX = i} ) = \frac{\frac{{CASE}_{i}}{CASE}}{\frac{{CASE}_{i} + {CONTROL}_{i}}{CASE + CONTROL}}$$

where CASEᵢ, is the fraction of the marker case group 106a (or subset 112 thereof) with SNP variant i; CONTROLᵢ is the fraction of the marker control group 106b (or subset 113 thereof) with SNP variant i; CASE is the total number of subjects within the marker data case group 106a (or subset 112 thereof); and CONTROL is the total number of subjects within the marker data control group 106b (or subset 113 thereof). For example, turning to FIG. 2, a propensity plotter module 216 is executed by the server 202 to determine propensity scores 222 related to the markers 110 associated with the genes 114.

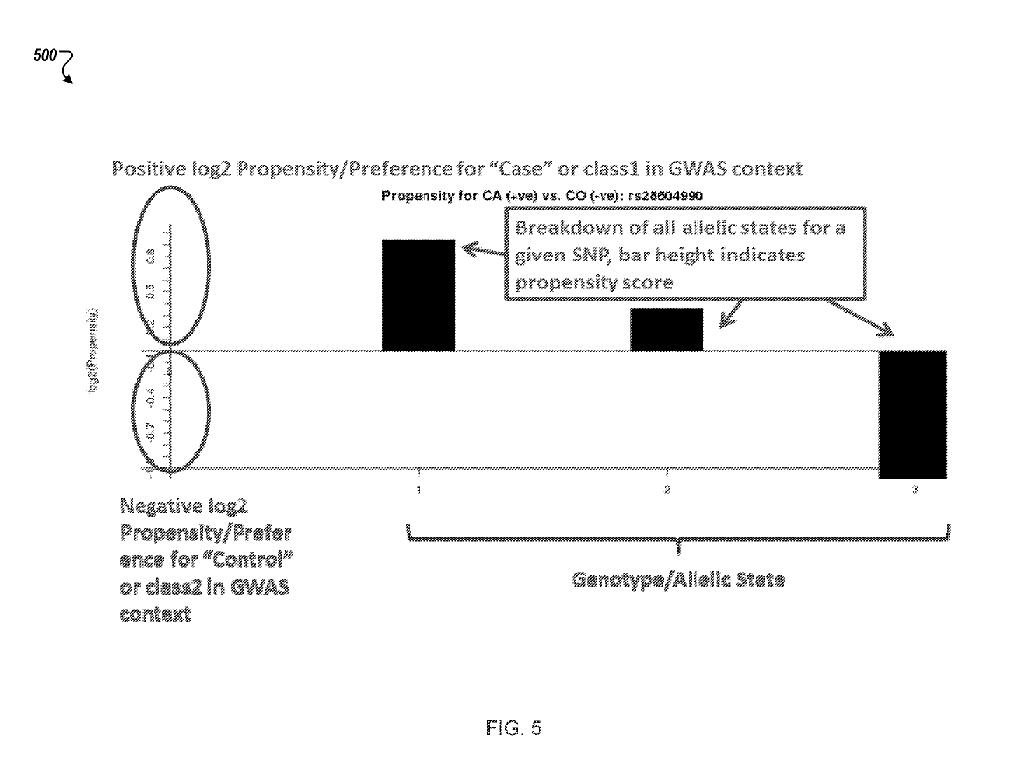

In some implementations, the propensity scores 222 are presented within a graphic interface. Turning to FIG. 5, a propensity plot 500 illustrates a breakdown of all allelic states for a given SNP. The bar height within the log 2 bar graph indicates the propensity score. A positive propensity score, in some implementations, indicates a propensity or preference for the case group marker data set 106a, while a negative propensity score indicates a propensity or preference for the control group marker data set 106b. The propensity score breakdown 116 enables a user to quickly distinguish allelic variants that have strong indication to be associated with the marker data case group 106a and/or the marker data control group 106b as established through analysis of the marker data 106 (e.g., GWAS).

Returning to FIG. 1, in some implementations, the propensity plot analysis results in refinement of the genes into a gene subset 118. A researcher, for example, may choose to focus on particular genes based upon review of the propensity scores 222 (e.g., via the propensity plot 500).

In some implementations, expression analysis is performed on a large dataset of gene expression data 120. Having identified the genes 114 (or the gene subset 118 thereof) as significant in both gene expression data and in marker data for at least a subset 112 of the marker data case group 106a, it is found that additional insight into the role of the identified genes 114 may be obtained by comprehensively searching genomic data sets to identify samples in which a same expression pattern occurs. A query method and algorithm may be used, for example, that takes as input a set of genes, and returns all samples from the large dataset of gene expression data where the input gene set is significantly upregulated or downregulated (or both). In a particular example, a researcher may want to target a specific pathway for upregulation in an experiment. The researcher may search for a particular profile using the query method and algorithm, limiting the result data to samples where the particular profile is upregulated. The samples outputted by the algorithm may then be browsed for conditions and/or treatments that upregulate the specific pathway. Similarly, a disease signature may be utilized to find other conditions and/or diseases whose expression profiles are similar to the disease of interest to find common pathways or common treatments.

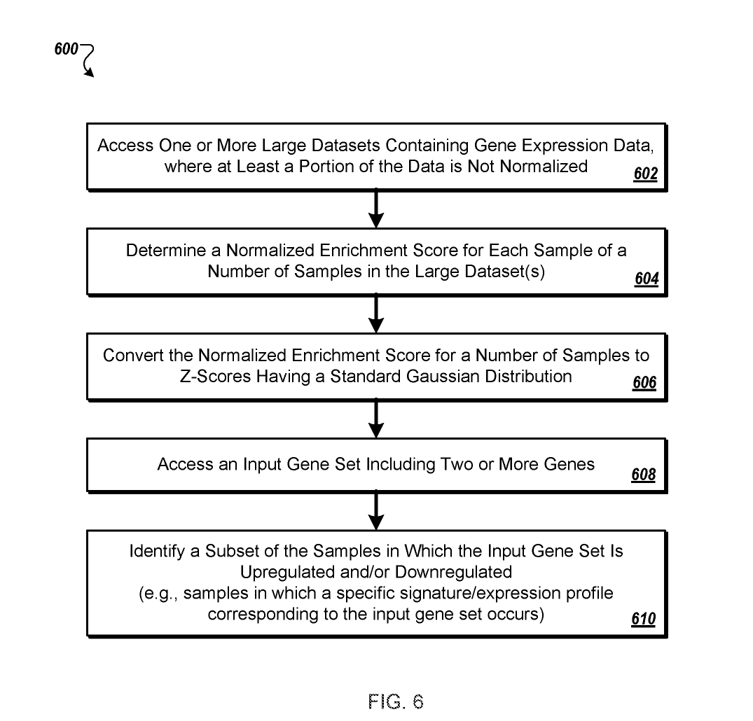

Turning to FIG. 2, in some implementations, an expression data search engine 218 is executed by the server 202 to perform expression analysis on a large dataset of gene expression data. The gene expression data, for example, may be downloaded from or accessed via one or more gene expression databases 204 available through a network connection. In a particular example, the records of the GEO database may be downloaded, and the expression data search engine module 218 may determine normalized enrichment scores for the downloaded records, as described in relation to a method 600 of FIG. 6.

Turning to FIG. 6, a flow chart of the method 600 is presented for use in searching for samples from a large dataset of gene expression data where the input gene set is significantly upregulated or downregulated (or both).

In some implementations, the method 600 begins with accessing one or more large datasets containing gene expression data (602). At least a portion of the data contained within the one or more large datasets is not normalized, such that expression data may not be directly queried. In a particular example, records from the GEO database of high throughput gene expression datasets may be accessed.

In some implementations, a normalized enrichment score is determined for each sample of a number of samples in the large dataset (604). A scoring function with several components may be used including, for example: a significance component (e.g., single-sample based Wilcox test function) to ascertain the significance of differential expression for probes annotated to a gene of interest against all other probes in the sample, a SNR component to identify an effect of size according the signal-to-noise ratio, and a difference component identifying the number of genes that are in the sample versus the number of genes in the full input set.

In a specific example, the normalized enrichment score (NES) is defined in Equation 1.

NES=(1−pval_{wilcox})*S2N*(|G|−|S|) (Equation 1)

As shown, pval_{wilcox} is the p-value from the single-sample based Wilcox test; S2N is the signal-to-noise ratio; |G| is the number of genes in the input; and |S| is the number of input genes in the sample (i.e., |Input Genes∩Genes in Sample|). The signal-to-noise ratio (S2N) is defined in Equation 2 in which μ is the mean, and sd is the standard deviation.

$$( Equation 2 )$$

$$S2N = \frac{{μ}_{rank} ( input\_genes \_in \_sample ) \times {μ}_{rank} ( other\_genes \_in \_sample )}{{sd}_{rank} ( input\_genes \_in \_sample ) + {sd}_{rank} ( other\_genes \_in \_sample )}$$

In some implementations, the normalized enrichment score (NES) of a number of samples is converted to z-scores having a standard Gaussian distribution (606). In such implementations, the NES follows a standard Gaussian distribution. This allows the scores to be converted to a z-scores with mean=“0” and sd=“1” and converted to a p-value using a standard Gaussian distribution function (e.g., pnorm in the R language).

In some implementations, an input gene set, including two or more genes, is accessed (608). The input gene set, for example, may be presented as query gene expression data to the large dataset including the normalized enrichment scores.

In some implementations, a subset of the samples of the large dataset in which the input gene set is upregulated and/or downregulated is identified (610). The subset of samples includes those samples in which a specific signature or expression profile corresponding to the input gene set occurs.

Returning to FIG. 1, in some implementations, the subset of samples corresponding to the input gene set is analyzed to identify a refined gene subset 122 of the gene subset 118. Alternatively, data discovered through expression analysis of the large dataset of gene expression data 120 is analyzed to identify subsets for grouping the second cohort 102b into the marker data case group subsets 112 and the marker data control group subsets 113, allowing for recursive performance of a portion of the flow diagram 100.

In some implementations, the genes 114, gene subset 118, or refined gene subset 122 (depending upon analysis performed in a particular study) is analyzed in light of targeted drug data 124 to identify one or more drug candidates 126 for restoring expression of the downregulated and/or upregulated genes. A drug data analyzer module 220, for example, may be executed by the server 202 of FIG. 2 to identify the one or more drug candidates 126. The drug data analyzer module 220 may access one or more drug databases 206 (e.g., via the Internet) or access locally stored drug database records previously collected from one or more public drug information databases. In some examples, the public drug information databases include the Cenla Medication Access Program (CMAP) maintained by the Broad Institute of the Massachusetts Institute of Technology and Harvard University of Boston, Mass.; the DrugBank database of the University of Alberta; the Genomics of Drug Sensitivity in Cancer Database (GDSC) maintained by the Sanger Institute of Hinxton, GB and the Massachusetts General Hospital Cancer Center of Boston, Mass.; or the drug annotation database records maintained by the National Cancer Institute of Rockville, Md. The identified drug candidates may in turn be assessed as potential treatments for subjects having the disease and/or condition of the case groups (104a, 106a).

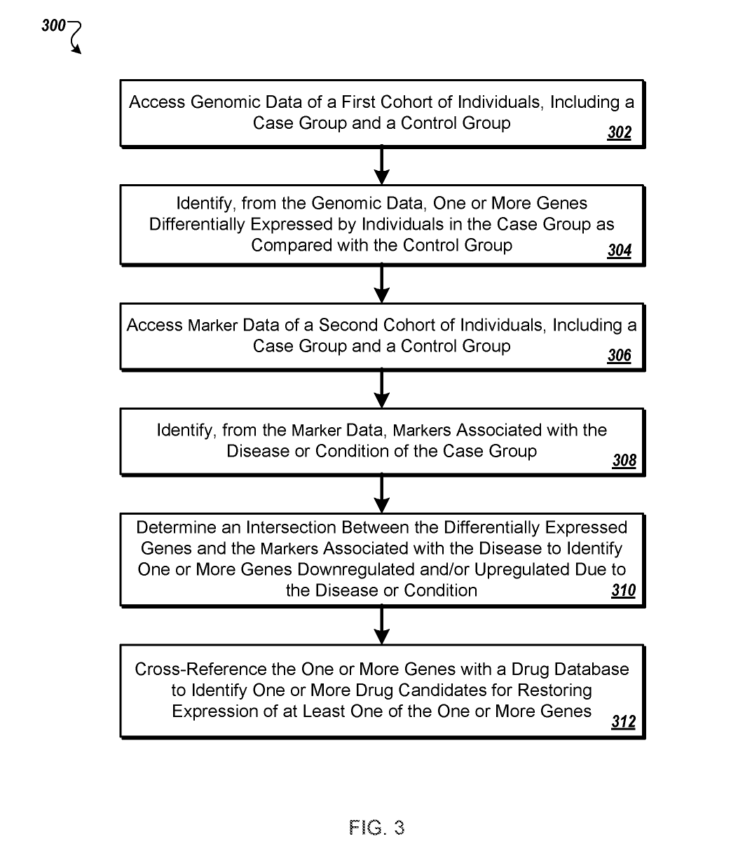

FIG. 3 is a flow chart of an example method 300 for identification of drug candidates for therapy of patients having a particular disease or condition based upon automated review of genomic data to identify downregulated and/or upregulated gene expression. The method 300, for example, may be performed at least in part by the server 202 described in relation to FIG. 2. For example, the differential expression identifier module 210, the marker association identifier module 212, the expression/association integrator module 214, and/or the drug data analyzer 220 may be used to perform portions of the method 300.

In some implementations, the method 300 begins with accessing genomic data of a first cohort of individuals, including a case group and a control group. The data may be accessed, for example, by the server 202 from one or more gene expression databases 204, as illustrated in FIG. 2. As described in relation to FIG. 1, the genomic data 104 of the first cohort 102a includes (a) the case group genomic data 104a, including samples related to individuals having a particular disease or condition, and (b) the control group genomic data 104b, including samples related to individuals who do not have the particular disease or condition.

In some implementations, one or more genes differentially expressed by individuals in the case group as compared with the control group are identified (304). The one or more genes, for example, may be identified as differentially expressed genes 108 by the differential expression identifier module 210 of FIG. 2. As described in relation to FIG. 1, the case group genomic data 104a can be compared to the control group genomic data 104b using a number of analysis tools, including the Linear Models for Microarray Data (LIMMA).

In some implementations, marker data of a second cohort of individuals, including a case group and a control group, is accessed (306). The data may be accessed, for example, by the server 202 from one or more gene expression databases 204, as illustrated in FIG. 2. As described in relation to FIG. 1, the marker data 106 of the second cohort 102b includes (a) the case group marker data 106a, including samples related to individuals having a particular disease or condition, and (b) the control group marker data 106b, including samples related to individuals who do not have the particular disease or condition. The individuals in the second cohort may overlap with the individuals in the first cohort, or the first cohort may include an entirely different population of individuals than the individuals of the second cohort.

In some implementations, markers associated with the disease or condition of the case group are identified from the marker data (308). The markers associated with the gene or condition 110, for example, may be identified by the marker association identifier module 212 of FIG. 2. For example, as described in relation to FIG. 1, a Genome Wide Association Study (GWAS) may be conducted on the case group marker data 106a and the control group marker data 106b to identify the markers 110.

In some implementations, an intersection between the differentially expressed genes and the markers associated with the disease is determined, and one or more genes downregulated and/or upregulated due to the disease or condition are identified (310). The genes downregulated and/or upregulated due to the disease or condition 114, for example, may be identified by the expression/association integrator module 214 of FIG. 2. For example, as described in relation to FIG. 1, one or more markers 110 may be identified as being near each of at least a subset of the differentially expressed genes 108.

In some implementations, one or more genes are cross-referenced with a drug database to identify one or more drug candidates for restoring expression of at least one of the one or more genes (312). The genes 114, for example, may be cross-referenced with information obtained from the drug database(s) 206 by the drug data analyzer module 220 of FIG. 2 to identify drug candidates 126.

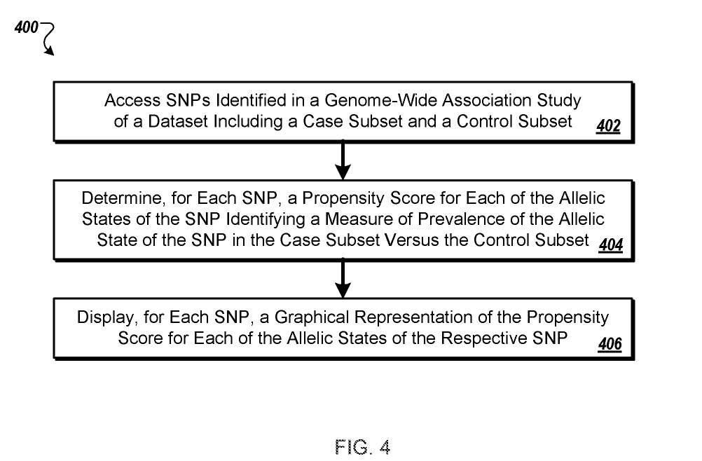

FIG. 4 is a flow chart of an example method 400 for determining and presenting propensity scores related to single-nucleotide polymorphisms. The method 400, for example, may be performed at least in part by the server 202 described in relation to FIG. 2. For example, the propensity plotter module 216 may be used to perform portions of the method 400.

In some implementations, the method 400 begins with accessing SNPs identified in a genome-wide association study of a dataset including a case subset and a control subset (402). The data may be accessed, for example, by the server 202 from one or more gene expression databases 204, as illustrated in FIG. 2. As described in relation to FIG. 1, the marker data 106 of the second cohort 102b includes (a) the case group marker data 106a, including samples related to individuals having a particular disease or condition and (b) the control group marker data 106b, including samples related to individuals who do not have the particular disease or condition.

In some implementations, for each allelic state of each SNP, a propensity score is determined (404). The propensity score identifies a measure of prevalence of the particular allelic state of the respective SNP in the case subset versus the control subset (404). A particular algorithm for propensity score calculation is provided in Equation 3.

$$log 2 ( {PROPENSITY}_{rsX = i} ) = \frac{\frac{{CASE}_{i}}{CASE}}{\frac{{CASE}_{i} + {CONTROL}_{i}}{CASE + CONTROL}} \qquad ( Equation 3 )$$

As shown, CASEᵢ is the fraction of the case group with SNP variant i; CONTROLᵢ is the fraction of the control group with SNP variant i; CASE is the total number of subjects within the case group; and CONTROL is the total number of subjects within the control group. For example, turning to FIG. 2, a propensity plotter module 216 may be executed by the server 202 to determine propensity scores 222 related to the markers associated with the genes 114.

In some implementations, a graphical representation of the propensity score for each of the allelic states of at least a first SNP is displayed (406). As described in relation to FIG. 5, the propensity plot 500 illustrates a breakdown of all allelic states for a given SNP in a bar graph format, where the bar height within the log 2 bar graph indicates the propensity score. Although illustrated in relation to a log 2 bar graph in FIG. 5, the propensity score graphical representation can include any visualization format which enables a user to quickly distinguish allelic variants that have strong indication to be associated with the case group and/or the control group as established through analysis of the SNP data (e.g., GWAS).

### Experimental Example

As exemplified below, the successful use of provided systems for identifying genetic features indicative of a particular disease or condition is presented. The systems are further used to identify drug candidates for treatment of the particular disease or condition. The example further describes identification of drug candidates likely to be effective in subsets of patients.

### Alzheimer Disease Study—Overview

One or more methods and systems of the present disclosure were applied to existing data from the Gene Expression Omnibus databases to identify genetic features and drug candidates for Alzheimer's disease (AD). The analysis combined the data from the gene expression and single nucleotide polymorphism (SNP) studies across different patient cohorts. The present AD study first identified AD-associated genes consistently altered with disease across a series of expression datasets. One or more methods and systems of the present disclosure were then employed to search publicly available microarray data. The search identified a link between one of the AD-associated genes (NEUROD6) and gender. In light of the finding and the observation of higher numbers of women having AD, the AD study stratified patients by both gender and APOE4 status. Multiple SNP datasets were analyzed to identify variants associated with AD. It was found that SNPs in the region of NEUROD6 were significantly associated with AD in APOE4+ females. It was also found that SNPs in the region of another AD-associated gene (SNAP25) were significantly associated with AD in APOE4+ males.

One or more methods and systems of the present disclosure were then employed to search for medicines that modulate these genes. The methods also identified subset-specific drug candidates. The results suggest that stratifying AD patients by gender and APOE4 status may yield additional targets and suggest new approaches for developing urgently needed treatments.

### Identification, Processing, and Analysis of Five Expression Datasets to Produce a Gene List

As indicated, to generate fresh insights into AD, one or more methods and systems of the present disclosure were applied to the GEO datasets and identified a list of genes significantly affected by Alzheimer's disease (AD). The expression datasets included the GEO database, including GEO project-accession numbers GSE5281, GSE1297, GSE36980, GSE15222, and GSE44772. Table 1 provides a summary of the expression datasets used in the present analysis, including the sample IDs for the samples used, the type of array the data was measured on, and the compartment of the brain that the samples were collected.

Each of the expression datasets was processed and analyzed separately. Table 2 provides a summary of the processing to each of the datasets.

TABLE 1
Summary of Expression Datasets Used in the Present Analysis

| | Brain | Total | |
|---|---|---|---|
| Dataset | Compartment | # of Samples | Array Type |
| GSE1297 | Hippocampus | 18 | Affymetrix Human Genome |
| | | | U133A Array |
| GSE5281 | Entorhinal Cortex | 23 | Affymetrix Human Genome |
| | (Isolated neurons) | | U133 Plus 2.0 Array |
| GSE15222 | Frontal Cortex, | 364 | Sentrix HumanRef-8 |
| | Temporal Cortex, | | Expression BeadChip |
| | Cerebellum, or | | |
| | Parietal Cortex | | |
| GSE36980 | Hippocampus | 17 | Affymetrix Human |
| | | | Gene 1.0 ST Array |
| GSE44772 | Prefrontal Cortex | 230 | Rosetta/Merck |
| | | | Human 44k |
| | | | 1.1 microarray |

TABLE 2
Summary of Processing to Individual Expression Dataset of Table 1

| Dataset | Samples | Notes |
|---|---|---|
| GSE1297 | GSM21204, GSM21206, | Compared samples |
| | GSM21207, GSM21209, | with top 9 NFT scores vs |
| | GSM21211-GSM21213, | bottom 9 NFT scores |
| | GSM21216, | |
| | GSM21218-GSM21222, | |
| | GSM21224, GSM21226, | |
| | GSM21230-GSM21232 | |
| GSE5281 | GSM119615-GSM119627, | Used only the EC samples, |
| | GSM238763, | compared AD (10 samples) |
| | GSM238790-GSM238798 | vs healthy (13 samples) |
| GSE15222 | All | Compared AD (176 samples) vs |
| | | healthy (188 samples) |
| GSE36980 | GSM907854-GSM907870 | Used only the Hippocampus |
| | | samples, compared AD |
| | | (7 samples) vs healthy (10 |
| | | samples) |
| GSE44772 | GSM1090501- | Used only the Prefrontal |
| | GSM1090730 | Cortex samples, |
| | | compared AD (129) vs. |
| | | healthy (101 |
| | | samples) |

The GSE5281 dataset included genomic data of samples collected from six compartments of the brains of patients with Alzheimer's disease (AD) and a control group. Details of the dataset are found in Liang, W. S. et al., “Altered Neuronal Gene Expression in Brain Regions Differentially Affected by Alzheimer's Disease: A Reference Data Set,” 33 Physiological Genomics 240-256 (2008). The samples collected from the Entorhinal Cortex (EC) were observed to have the highest quality dataset within the six compartments and were the only subset used within the present analysis. Raw data from the EC samples was obtained from the GEO database in the form of CEL files for both the AD patients and the control group. The raw data was RMA normalized, and Linear Models for Microarray Data (LIMMA) were used, for example, to determine the probes significantly different between the AD patients and the control group (in which the multiple hypothesis corrected p-value is less than 0.05). The results were filtered, for example, using Presence/Absence calls computed in R using the “affy” package. Probes that were downregulated in the AD patients compared to the control group were filtered in which the filtering required that the average Presence/Absence call for all of the control samples are at least marginal. Additionally, probes that were upregulated in the AD patients compared to the control group were also filtered in which the filtering required that the average Presence/Absence call for all of the AD patient samples are at least marginal. Examples of LIMMA are described in Smyth, G. K., “Linear Models and Empirical Bayes Methods for Assessing Differential Expression in Microarray Experiments,” Stat Appl Genet Mol Biol, Vol. 3 (2004). Examples of the affy package are described in Gautier, L. et al. “affy—Analysis of Affymetrix GeneChip Data at the Probe Level,” 20 Bioinformatics 307-315 (2004).

The GSE1297 dataset included AD information (e.g., presence of AD), mini-mental state examination (MMSE) scores, and neurofibrillary tangle (NFT) scores for each individual in the dataset. Details of the GSE1297 dataset are found in Blalock, E. M. et al., “Incipient Alzheimer's Disease: Microarray Correlation Analyses Reveal Major Transcriptional and Tumor Suppressor Responses,” 101.7 Proceedings of the National Academy of Sciences of the United States of America 2173-2178 (2004). The dataset was analyzed a number of different ways, and it was found that (a) comparing data of individuals with the 9 highest NFT scores versus the data of individuals with the 9 lowest NFT scores generated approximately five times more significant probes than (b) comparing Severe AD patients (identified based on the MMSE score) versus the control group. To this end, samples with the top 9 NFT scores versus the lowest 9 NFT scores were used in the present analysis. In the dataset, the lowest 9 NFT scores included both the control group and patients labeled as having incipient AD; the highest 9 NFT scores included patients labeled as having severe AD, moderate AD, and incipient AD. The CEL files for the samples were also obtained from GEO database. The data was RMA normalized; and probes significantly different in the two conditions were determined using LIMMA. The results were also filtered according to Presence/Absence calls in the same manner as the GSE5281 dataset.

The GSE36980 dataset included genomic data of samples collected from three compartments of the brains of AD patients and a control group. Details of the dataset are found in Nakabeppu Y. et al., “Expression Data From Post Mortem Alzheimer's Disease Brains,” pmid. GSE36980 (2013). The data from the hippocampus was observed to have the most number of significant probes when comparing the AD patients to the control group and was used in the analysis. The CEL files for these samples were obtained from GEO database. The data was RMA normalized, and the probes significantly different in the two conditions were also determined using LIMMA.

The GSE15222 dataset included genomic data of samples collected from a number of compartments of the brains of AD patients and a control group. Details of the dataset are found in Webster, J. A. et al., “Genetic Control of Human Brain Transcript Expression in Alzheimer Disease,” The American Journal of Human Genetics, Volume 84, Issue 4, 445-458 (2009). Rather than raw data, processed data in that study were available. The dataset was obtained from the corresponding author's lab website and was found to be rank-invariant normalized and filtered in two ways—first, transcripts were only considered if they were detected in at least 90% of the AD patients or 90% of the control group, and second, the transcript expression intensities were only considered in the analyses for a given sample if their Illumina detections scores were greater than 0.99. Since the data had been normalized across all measured compartments of the brain, all the samples of the GSE36980 dataset were considered in the present analysis. LIMMA was also employed to determine the probes significantly different between the AD patients and the control group.

The GSE44772 dataset included genomic data of samples collected from three compartments in the brains of AD patients and a control group. The data from the prefrontal cortex were used for the analysis. Rather than raw data, processed data were available from that study in the Series Matrix File of the GEO database. The data were in the form of normalized log 10 ratios between the test sample and a pooled reference sample. LIMMA was also used to determine the probes that were significantly different between the AD patients and the control group.

The analyses in the present AD study generated a list of probes significantly affected by Alzheimer's disease for each of the five datasets. To obtain a robust list of affected genes, one or more methods and systems of the present disclosure were employed to determine intersections of the gene names from all five datasets. Separate intersections for genes upregulated in each study and genes downregulated were performed in each study.

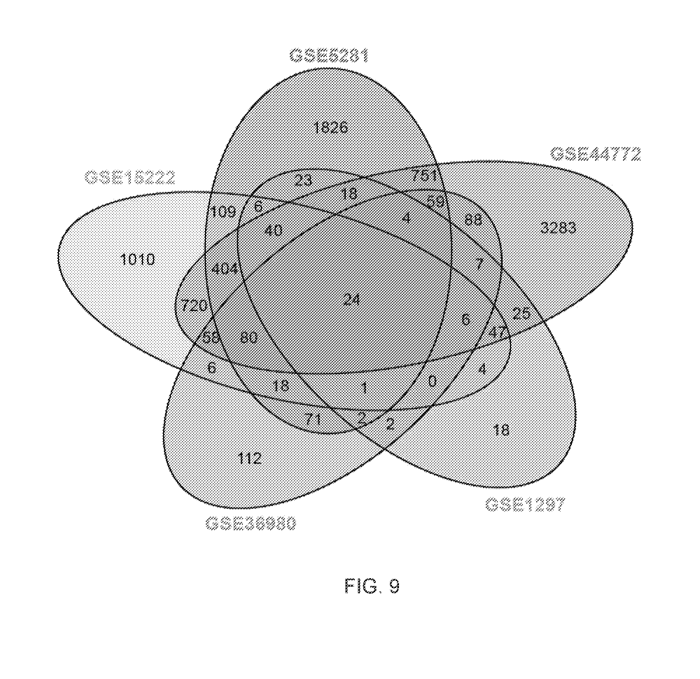

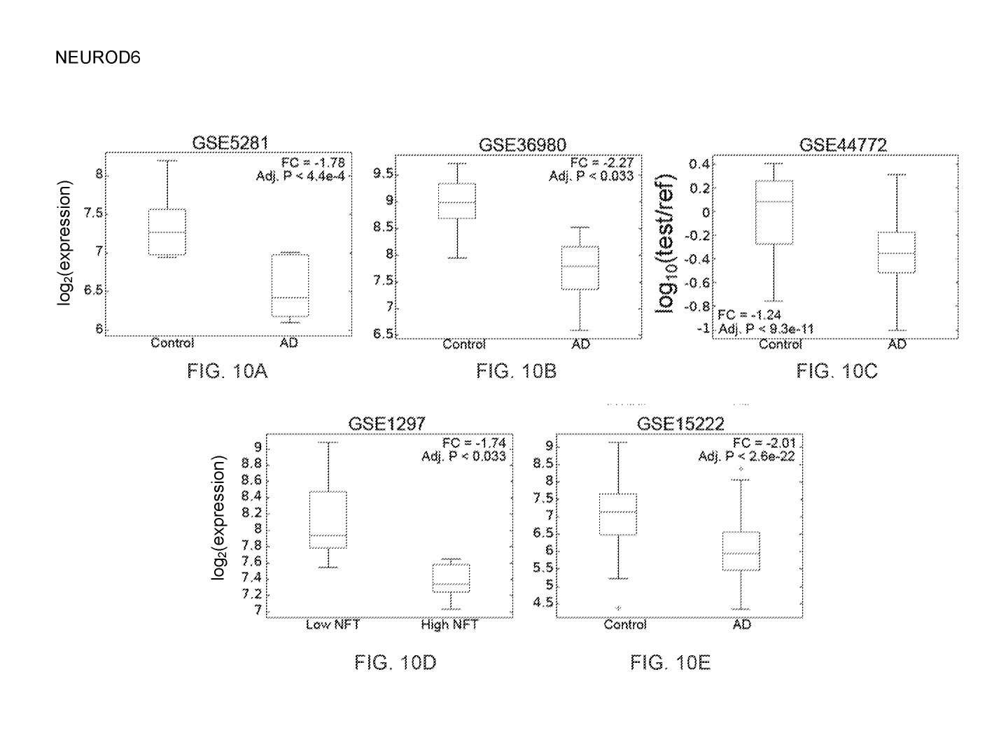

While every study may have limitations and may have inherent noise in the data, it was reasoned that signals appearing consistently across multiple datasets would be more robust. To this end, genes with significantly different expression levels in healthy controls and AD patients (as defined by overall diagnosis or NFT score) in each of the five expression datasets were identified. Genes observed to be downregulated in AD consistently across the five datasets are shown in relation to FIGS. 9, 10(A-E), and 16. Taking the intersection, the analysis identified 24 genes that were significantly downregulated with disease in all five datasets.

FIG. 9 is an example Venn diagram illustrating intersections of significantly downregulated genes across the five datasets of the AD study. Table 3 shows the 24 identified genes.

TABLE 3
Identified 24 Downregulated Genes from the Five Datasets

AP3B2
ATP1A3
ATP5B
ATP6V1E1
ATP6V1G2
BNIP3
C14orf132
C14orf2
CACNG3
GNG3
GOT2
MAGED1
MRPS11
NEUROD6
PPP1R11
PTPRN2
RGS7
SLC17A7
SLC25A11
SNAP25
SYP
TPI1
UQCRC1
YWHAB

FIGS. 10A-10E show box plots of NEUROD6 expression, activity, or combination illustrating the consistent downregulation of the identified genes across each of the respective GSE5281, GSE1297, GSE36980, GSE15222, and GSE44772 datasets.

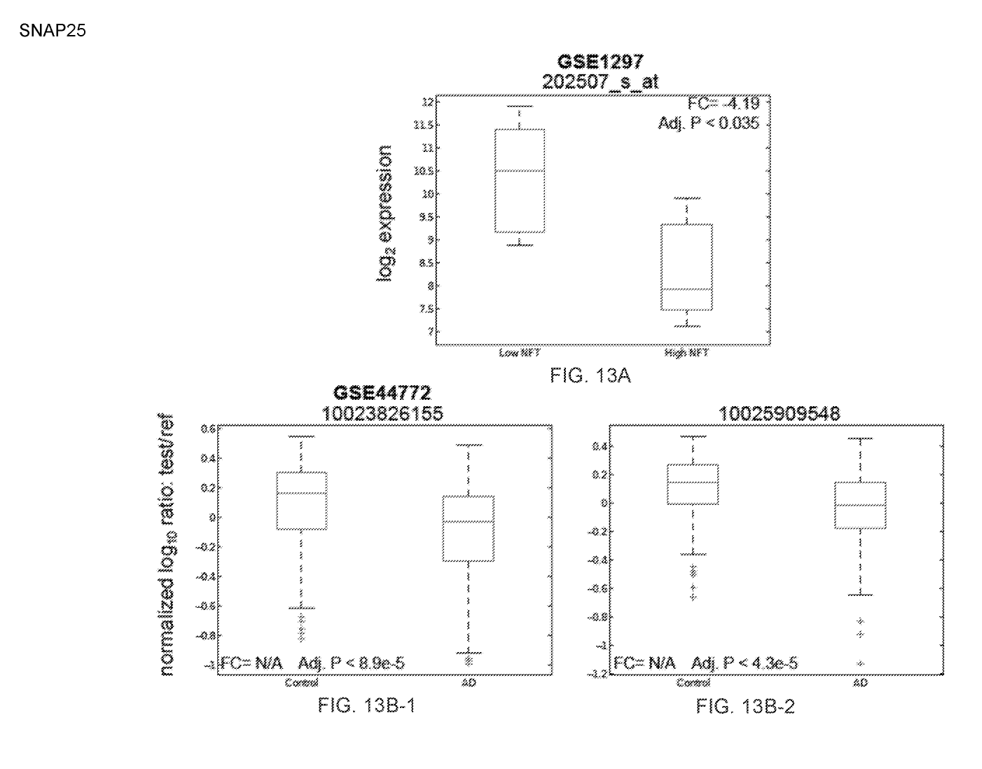

FIGS. 13A, 13B-1, 13B-2, 13C-1, 13C-2, 13D, and 13E show box plots of SNAP25 expression, activity, or combination illustrating the consistent downregulation of the identified genes across each of the respective GSE5281, GSE1297, GSE36980, GSE15222, and GSE44772 datasets. Three probesets were present for measuring SNAP25 expression, and plots are shown for each. The plots show significantly disease-associated SNPs near SNAP25 in APOE4+ male patients, but not in APOE4+ female patients or APOE4− male patients. Such SNPs related to SNAP25 in APOE4+ male patients (in the LOAD and Cell datasets, respectively) include (a) rs6077693 (p<0.00029 in male APOE4+, p<0.7257 in female APOE4+, and p<0.7328 in male APOE4−) and (b) rs6032806 (p<0.00043 in male APOE4+, p<0.6579 in female APOE4+, and p<0.6948 in male APOE4−). Probeset 202508_s_at exclusively targets the 3′ UTR region rather than the coding region of the SNAP25 gene, whereas the other two probesets contain probes matching regions in the exons. Since detection of mRNA based on 3′ UTR probesets becomes less accurate for genes that have alternative polyadenylation sites in different tissues, and SNAP25 contains multiple alternative polyadenylation sites in brain different from those in other tissues, the probeset 202508_s_at results may be less reflective of true SNAP25 mRNA expression levels.

Additional details on fold changes and p-values for the 24 genes for each of the datasets are provided in Table 4.

TABLE 4
Summary of Fold Changes and p-values for the 24 Genes for each of the Five Expression Datasets

| | GSE5281 | | | GSE1297 | | | GSE36980 | | |
|---|---|---|---|---|---|---|---|---|---|
| Gene Symbol | FC | P. Value | adj. P. Val | FC | P. Value | adj. P. Val | FC | P. Value | adj. P. Val |
| AP3B2 | −2.33 | 1.08E−05 | 3.17E−04 | −1.79 | 3.46E−04 | 3.54E−02 | −1.46 | 3.85E−04 | 3.65E−02 |
| ATP1A3 | −4.36 | 6.92E−07 | 5.15E−05 | −3.22 | 5.97E−04 | 4.09E−02 | −1.47 | 2.65E−04 | 3.43E−02 |
| ATP5B | −3.13 | 2.43E−04 | 2.96E−03 | −1.91 | 5.09E−04 | 3.95E−02 | −1.30 | 5.96E−04 | 3.97E−02 |
| ATP6V1E1 | −2.26 | 1.62E−03 | 1.18E−02 | −1.75 | 6.04E−04 | 4.09E−02 | −1.37 | 6.88E−05 | 2.42E−02 |
| ATP6V1G2 | −3.56 | 1.52E−04 | 2.10E−03 | −2.81 | 1.16E−04 | 2.91E−02 | −1.58 | 5.71E−05 | 2.42E−02 |
| BNIP3 | −2.04 | 3.14E−05 | 6.77E−04 | −1.97 | 8.41E−04 | 4.56E−02 | −1.28 | 3.10E−04 | 3.44E−02 |
| C14orf132 | −2.12 | 1.17E−04 | 1.73E−03 | −1.37 | 4.85E−04 | 3.86E−02 | −1.27 | 4.24E−04 | 3.69E−02 |
| C14orf2 | −2.27 | 1.46E−04 | 2.04E−03 | −1.53 | 3.62E−04 | 3.57E−02 | −1.39 | 6.63E−04 | 4.13E−02 |
| CACNG3 | −3.35 | 1.29E−06 | 7.59E−05 | −2.19 | 1.38E−04 | 2.98E−02 | −1.92 | 4.62E−04 | 3.76E−02 |
| GNG3 | −4.60 | 8.67E−06 | 2.72E−04 | −1.90 | 1.78E−04 | 3.18E−02 | −1.51 | 7.17E−04 | 4.19E−02 |
| GOT2 | −2.31 | 7.63E−04 | 6.77E−03 | −1.56 | 6.62E−05 | 2.62E−02 | −1.37 | 5.37E−04 | 3.90E−02 |
| MAGED1 | −2.04 | 1.06E−02 | 4.61E−02 | −1.85 | 5.20E−04 | 3.95E−02 | −1.29 | 4.03E−04 | 3.66E−02 |
| MRPS11 | −1.80 | 6.76E−04 | 6.19E−03 | −1.29 | 7.00E−04 | 4.25E−02 | −1.27 | 9.58E−05 | 2.61E−02 |
| NEUROD6 | −1.78 | 1.67E−05 | 4.36E−04 | −1.74 | 2.99E−04 | 3.33E−02 | −2.27 | 2.25E−04 | 3.29E−02 |
| PPP1R11 | −2.83 | 1.54E−05 | 4.11E−04 | −1.22 | 2.36E−04 | 3.32E−02 | −1.27 | 4.71E−06 | 1.46E−02 |
| PTPRN2 | −1.89 | 6.20E−03 | 3.14E−02 | −2.00 | 1.64E−04 | 3.18E−02 | −1.43 | 2.74E−04 | 3.43E−02 |
| RG57 | −2.65 | 9.45E−05 | 1.48E−03 | −2.32 | 1.58E−04 | 3.17E−02 | −1.63 | 2.33E−04 | 3.30E−02 |
| SLC17A7 | −4.59 | 2.79E−08 | 8.14E−06 | −2.64 | 3.00E−04 | 3.33E−02 | −1.54 | 4.29E−04 | 3.70E−02 |
| SLC25A11 | −1.65 | 4.39E−04 | 4.54E−03 | −1.58 | 9.79E−04 | 4.77E−02 | −1.26 | 1.18E−03 | 4.88E−02 |
| SNAP25 | −2.05 | 5.94E−05 | 1.06E−03 | −4.19 | 3.34E−04 | 3.49E−02 | −1.51 | 4.28E−04 | 3.70E−02 |
| SYP | −2.87 | 5.72E−08 | 1.15E−05 | −1.90 | 2.92E−04 | 3.33E−02 | −1.43 | 3.03E−04 | 3.44E−02 |
| TPI1 | −3.91 | 1.27E−06 | 7.53E−05 | −1.62 | 2.66E−04 | 3.33E−02 | −1.47 | 7.29E−06 | 1.46E−02 |
| UQCRC1 | −2.58 | 2.92E−03 | 1.81E−02 | −1.70 | 7.12E−04 | 4.25E−02 | −1.18 | 4.12E−04 | 3.69E−02 |
| YWHAB | −3.53 | 1.24E−03 | 9.68E−03 | −2.43 | 4.15E−04 | 3.74E−02 | −1.32 | 9.69E−04 | 4.59E−02 |

| | GSE15222 | | | GSE44772 | | |
|---|---|---|---|---|---|---|
| Gene Symbol | FC | P. Value | adj. P. Val | FC | P. Value | adj. P. Val |
| AP3B2 | −1.15 | 1.73E−06 | 5.31E−06 | NA | 2.70E−05 | 1.26E−04 |
| ATP1A3 | −1.19 | 7.57E−08 | 2.96E−07 | NA | 2.77E−08 | 4.38E−07 |
| ATP5B | −1.14 | 1.25E−09 | 6.93E−09 | NA | 3.11E−06 | 2.00E−05 |
| ATP6V1E1 | −1.36 | 3.90E−16 | 1.01E−14 | NA | 1.33E−07 | 1.51E−06 |
| ATP6V1G2 | −1.33 | 2.39E−11 | 1.95E−10 | NA | 3.41E−06 | 2.16E−05 |
| BNIP3 | −1.13 | 3.45E−08 | 1.45E−07 | NA | 8.90E−07 | 7.00E−06 |
| C14orf132 | −1.08 | 2.43E−03 | 4.29E−03 | NA | 2.22E−07 | 2.27E−06 |
| C14orf2 | −1.42 | 2.52E−24 | 1.28E−21 | NA | 8.28E−07 | 6.60E−06 |
| CACNG3 | −1.55 | 9.66E−15 | 1.77E−13 | NA | 1.88E−06 | 1.31E−05 |
| GNG3 | −1.34 | 9.46E−19 | 5.28E−17 | NA | 1.77E−09 | 6.16E−08 |
| GOT2 | −1.16 | 5.71E−07 | 1.90E−06 | NA | 1.35E−06 | 9.98E−06 |
| MAGED1 | −1.29 | 1.23E−14 | 2.19E−13 | NA | 3.28E−09 | 9.47E−08 |
| MRPS11 | −1.14 | 9.18E−04 | 1.75E−03 | NA | 9.12E−08 | 1.12E−06 |
| NEUROD6 | −2.01 | 4.18E−25 | 2.58E−22 | NA | 3.29E−14 | 9.22E−11 |
| PPP1R11 | −1.11 | 5.78E−08 | 2.31E−07 | NA | 6.04E−05 | 2.54E−04 |
| PTPRN2 | −1.43 | 7.36E−15 | 1.38E−13 | NA | 2.50E−08 | 4.06E−07 |
| RG57 | −1.39 | 3.06E−09 | 1.57E−08 | NA | 1.54E−08 | 2.85E−07 |
| SLC17A7 | −1.59 | 1.51E−09 | 8.22E−09 | NA | 1.21E−06 | 9.00E−06 |
| SLC25A11 | −1.12 | 2.37E−06 | 7.08E−06 | NA | 4.40E−06 | 2.66E−05 |
| SNAP25 | −1.41 | 5.75E−08 | 2.30E−07 | NA | 7.72E−06 | 4.30E−05 |
| SYP | −1.47 | 2.18E−14 | 3.69E−13 | NA | 1.83E−04 | 6.68E−04 |
| TPI1 | −1.30 | 1.80E−06 | 5.47E−06 | NA | 1.83E−04 | 6.68E−04 |
| UQCRC1 | −1.11 | 9.54E−05 | 2.14E−04 | NA | 5.20E−08 | 7.18E−07 |
| YWHAB | −1.08 | 4.55E−03 | 7.64E−03 | NA | 5.83E−06 | 3.39E−05 |

While establishing such a criterion may eliminate some relevant genes from consideration in the present AD study, it was reasoned that the resulting identified genes would be unambiguously associated with AD.

### NeuroD6 Brain Specificity Heat Maps

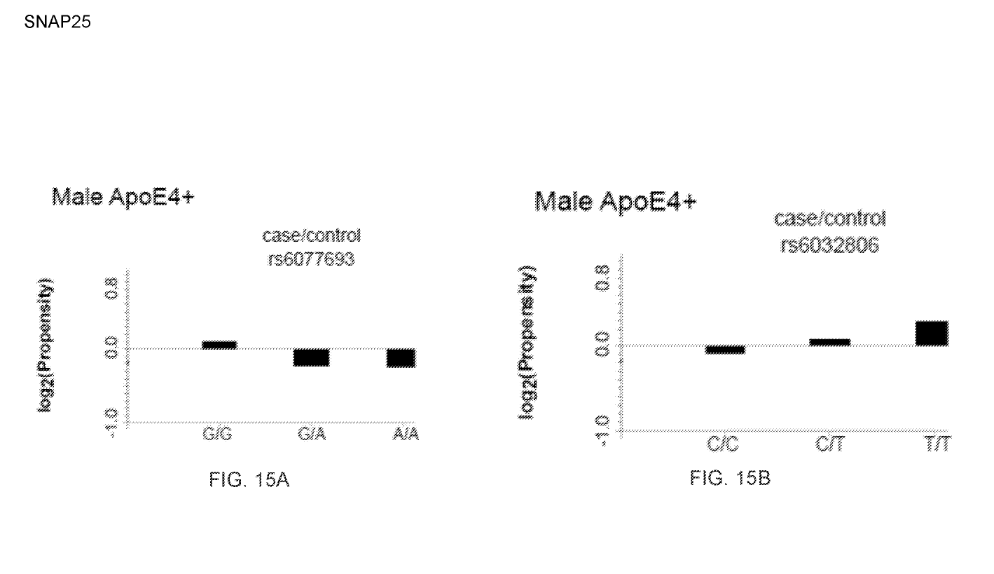

For the 24 genes identified, it was observed that a high degree of specificity exists for NEUROD6 expression in brain tissue. FIG. 16 illustrates a specificity heat map of NEUROD6 in brain tissue. For the 24 genes of interest, the probes were mapped to tissue specific arrays, available via BioGPS. Both outputs were displayed via a matrix visualization and analysis platform. Details of the BioGPS are found in Wu, C. et al., “BioGPS: An Extensible and Customizable Portal for Querying and Organizing Gene Annotation Resources,” 10 Genome Biology R130 (2009). As shown in the figure, the probes are clustered hierarchically with a metric of 1-Pearson correlation and displayed after being subtracted by the median and divided by the absolute deviation. Tissues are shown annotated by whether or not they are brain-associated, and sorted and grouped accordingly.

### Stratified Genome-Wide Association Studies (GWAS)

To generate additional insight into the role of NEUROD6, the methods and systems of the present disclosure were employed to comprehensively search all of the 500,000+ human datasets in the National Institutes of Health Gene Expression Omnibus (NIH GEO) to identify samples in which NEUROD6 was significantly overexpressed relative to other genes. A standard single-sample Wilcox test was employed to ascertain the significance of differential expression for probes annotated to a gene of interest against all other probes in the sample. The test was corrected for FDR, for example, using the Benjamini-Hochberg method in which the p-values were adjusted for multiple hypothesis testing across the full database. Based on the analysis, 38 samples from the healthy brain data from the GSE11882 dataset were found to be significantly enriched (FDR adjusted p.value <0.05) for high expressions of NEUROD6. Table 5 shows patient samples identified to have high or enriched NEUROD6 expression from the dataset of healthy controls. The table shows the p-values and adjusted p-values for each of the identified samples.

TABLE 5
Patient Samples Identified to have High or Enriched NEUROD6
Expression from the Dataset of Healthy Controls

| Sample ID | Sample Name | Gender | Dataset | p.val | adj.p.val |
|---|---|---|---|---|---|
| GSM300282 | SuperiorFrontalGyrus_male_20yrs_indiv78 | M | GSE11882 | 0 | 0.00E+00 |
| GSM300309 | Hippocampus_male_22yrs_indiv85 | M | GSE11882 | 0 | 0.00E+00 |
| GSM300270 | SuperiorFrontalGyrus_male_86yrs_indiv73 | M | GSE11882 | 7.38E−13 | 8.01E−10 |
| GSM300254 | SuperiorFrontalGyrus_male_21yrs_indiv66 | M | GSE11882 | 3.80E−12 | 3.92E−09 |
| GSM300286 | Hippocampus_male_69yrs_indiv8 | M | GSE11882 | 3.80E−12 | 3.92E−09 |
| GSM300307 | SuperiorFrontalGyrus_male_33yrs_indiv84 | M | GSE11882 | 4.38E−12 | 4.49E−09 |
| GSM300275 | EntorhinalCortex_male_20yrs_indiv77 | M | GSE11882 | 1.52E−10 | 1.45E−07 |
| GSM300246 | PostcentralGyrus_male_70yrs_indiv53 | M | GSE11882 | 1.79E−10 | 1.70E−07 |
| GSM300260 | SuperiorFrontalGyrus_male_40yrs_indiv68 | M | GSE11882 | 1.28E−09 | 1.18E−06 |
| GSM300266 | SuperiorFrontalGyrus_male_75yrs_indiv72 | M | GSE11882 | 3.21E−09 | 2.93E−06 |
| GSM300298 | Hippocampus_female_30yrs_indiv82 | F | GSE11882 | 3.37E−09 | 3.06E−06 |
| GSM300176 | SuperiorFrontalGyrus_male_45yrs_indiv12 | M | GSE11882 | 4.07E−09 | 3.64E−06 |
| GSM300175 | PostcentralGyrus_male_45yrs_indiv12 | M | GSE11882 | 3.36E−08 | 2.89E−05 |
| GSM300339 | Hippocampus_female_82yrs_indiv98 | F | GSE11882 | 5.79E−08 | 4.88E−05 |
| GSM300319 | SuperiorFrontalGyrus_male_45yrs_indiv87 | M | GSE11882 | 7.65E−08 | 6.31E−05 |
| GSM300258 | EntorhinalCortex_male_40yrs_indiv68 | M | GSE11882 | 1.86E−07 | 1.46E−04 |
| GSM300315 | SuperiorFrontalGyrus_male_42yrs_indiv86 | M | GSE11882 | 1.94E−07 | 1.52E−04 |
| GSM300317 | Hippocampus_male_45yrs_indiv87 | M | GSE11882 | 2.06E−07 | 1.61E−04 |
| GSM300264 | SuperiorFrontalGyrus_male_52yrs_indiv71 | M | GSE11882 | 4.25E−07 | 3.21E−04 |
| GSM300235 | Hippocampus_male_85yrs_indiv46 | M | GSE11882 | 5.37E−07 | 3.95E−04 |
| GSM300293 | EntorhinalCortex_female_48yrs_indiv81 | F | GSE11882 | 5.86E−07 | 4.26E−04 |
| GSM300211 | SuperiorFrontalGyrus_male_28yrs_indiv29 | M | GSE11882 | 7.03E−07 | 4.99E−04 |
| GSM300278 | SuperiorFrontalGyrus_male_20yrs_indiv77 | M | GSE11882 | 1.21E−06 | 8.05E−04 |
| GSM300316 | EntorhinalCortex_male_45yrs_indiv87 | M | GSE11882 | 1.95E−06 | 1.20E−03 |
| GSM300281 | PostcentralGyrus_male_20yrs_indiv78 | M | GSE11882 | 2.35E−06 | 1.42E−03 |
| GSM300204 | EntorhinalCortex_male_83yrs_indiv28 | M | GSE11882 | 8.78E−06 | 4.90E−03 |
| GSM300296 | SuperiorFrontalGyrus_female_48yrs_indiv81 | F | GSE11882 | 9.29E−06 | 5.16E−03 |
| GSM300310 | PostcentralGyrus_male_22yrs_indiv85 | M | GSE11882 | 9.42E−06 | 5.22E−03 |
| GSM300206 | PostcentralGyrus_male_83yrs_indiv28 | M | GSE11882 | 1.09E−05 | 5.93E−03 |
| GSM300292 | SuperiorFrontalGyrus_female_44yrs_indiv80 | F | GSE11882 | 1.12E−05 | 6.06E−03 |
| GSM300289 | EntorhinalCortex_female_44yrs_indiv80 | F | GSE11882 | 1.19E−05 | 6.46E−03 |
| GSM300207 | SuperiorFrontalGyrus_male_83yrs_indiv28 | M | GSE11882 | 2.57E−05 | 1.32E−02 |
| GSM300320 | EntorhinalCortex_female_47yrs_indiv88 | F | GSE11882 | 3.43E−05 | 1.69E−02 |
| GSM300269 | PostcentralGyrus_male_86yrs_indiv73 | M | GSE11882 | 4.10E−05 | 1.96E−02 |
| GSM300299 | SuperiorFrontalGyrus_female_30yrs_indiv82 | F | GSE11882 | 4.50E−05 | 2.13E−02 |
| GSM300304 | EntorhinalCortex_male_33yrs_indiv84 | M | GSE11882 | 4.84E−05 | 2.24E−02 |
| GSM300259 | PostcentralGyrus_male_40yrs_indiv68 | M | GSE11882 | 4.87E−05 | 2.26E−02 |
| GSM300303 | SuperiorFrontalGyrus_male_20yrs_indiv83 | M | GSE11882 | 6.73E−05 | 2.97E−02 |

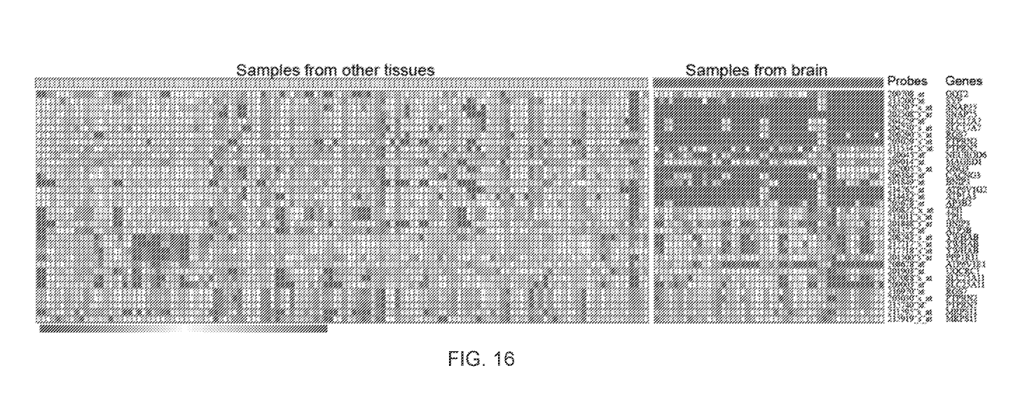

Strikingly, it was found that 30 of the 38 samples with enriched NEUROD6 expression were from males. This difference was highly significant, with hypergeometric p-value 1.71×10⁻⁴ and suggested a link between NEUROD6 and gender. FIG. 17 shows a plot of the distribution of the 38 samples between the male and female population with enriched NEUROD6 expression. In contrast, the entire GSE11882 dataset was well-balanced by gender (173 samples with 82 female and 91 male).

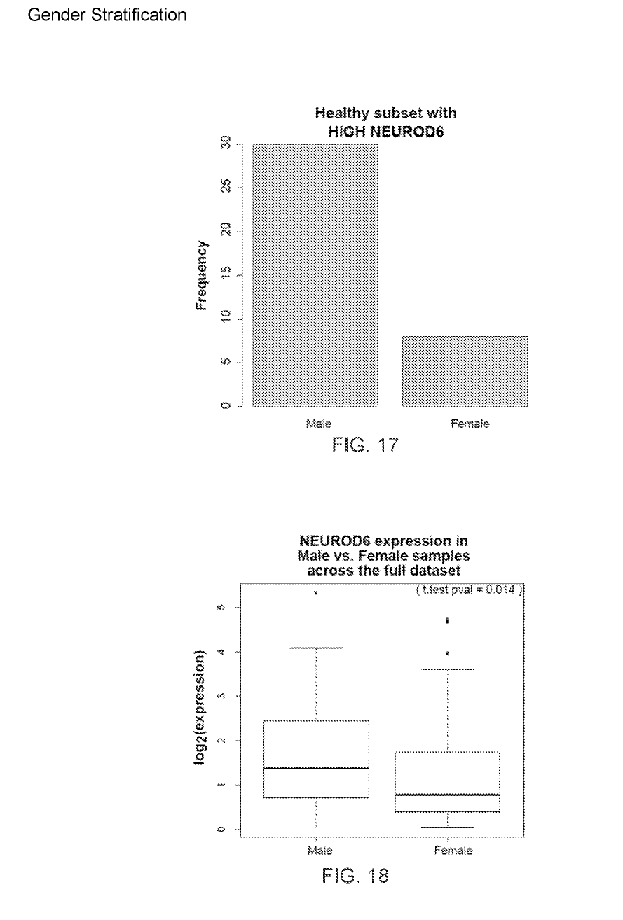

Subsequently, the pattern of NEUROD6 expression was analyzed across the datasets to search for expression differences by gender in NEUROD6. It was found that NEUROD6 expression was significantly higher in males than females with a nominal p value of 0.014. FIG. 18 illustrates a comparison of NEUROD6 expression by gender across the entire dataset of healthy controls.

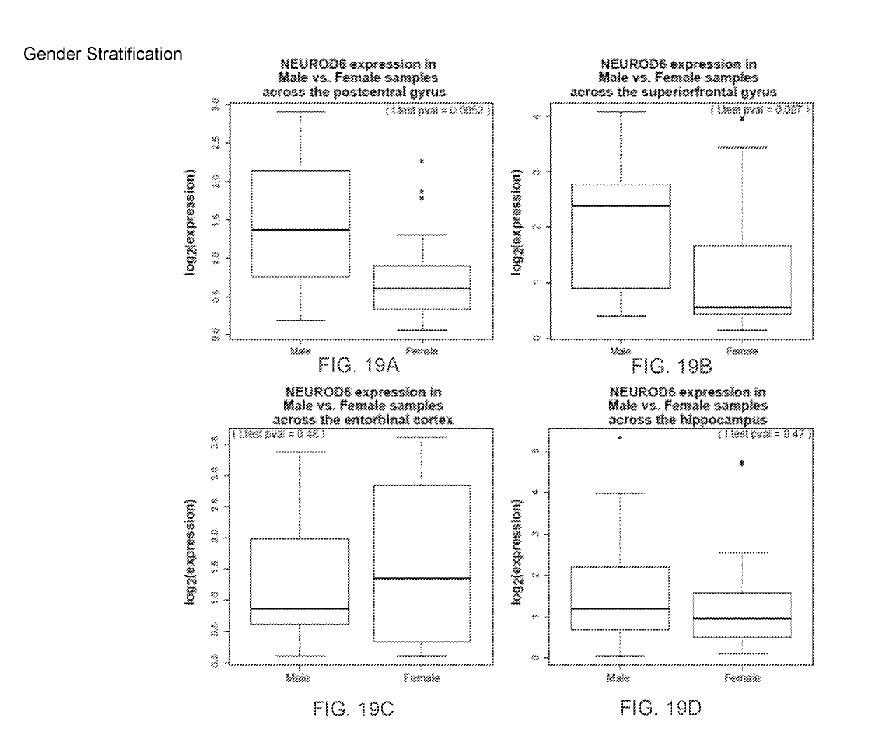

By dividing the dataset into individual compartments, it was found that NEUROD6 was significantly differentially expressed in two of the four compartments (with nominal p-values 0.0052 and 0.007), as shown in FIGS. 19A-D. The finding that NEUROD6 differs in expression level between healthy men and women is particularly intriguing given that (a) NEUROD6 expression is downregulated with disease, and that (b) gender may play a role in AD. Repeating the analysis with SNAP25, it was found that for two out of three expression probes, SNAP25 was significantly differentially expressed in an individual compartment in the GSE11882 dataset (nominal p-values 0.018 and 0.032). Table 6 illustrates the p-values for SNAP25 gene expression probes in male versus female samples in the GSE11882 dataset.

TABLE 6
P-values for SNAP25 Gene Expression Probes in Male Versus Female
Samples in the GSE11882 dataset

| | | Entorhinal | | Postcentral | Superiorfrontal |
|---|---|---|---|---|---|
| | Full Dataset | Cortex | Hippocampus | Gyrus | Gyrus |
| 202507_a_at | 0.13 | 0.31 | 0.85 | 0.052 | 0.032 |
| 202508_s_at | 0.088 | 0.68 | 0.46 | 0.25 | 0.018 |
| 1556629_a_at | 0.2 | 0.89 | 0.43 | 0.23 | 0.15 |

The significant p values are observed for the probesets 202507_a_at and 202508_s_at in the Superiorfrontal gyms; the p values are observed to be trending towards significance for the probeset 202507_a_at in the Postcentral gyms and the probeset 202508_s_at in the full dataset.

To distinguish between downstream signals resulting from disease pathology, and upstream signals that may be more causative and therefore better targets for therapy, single-nucleotide polymorphism (SNP) data in conjunction with the gene expression data were used. The established method was not used for combining these two types of data (e.g., the expression quantitative trait locus (eQTL) analysis) because that analysis requires both gene expression and SNP data to be from the same cohort of patients. Since most of the available gene expression and SNP data came from separate cohorts, one or more methods and systems of the present disclosure were employed to identify converging lines of evidence for disease-causing genes from both types of data.

Subsequently, regions in and around the 24 genes, identified in relation to Table 3, were examined for disease-associated SNPs in three datasets: the Alzheimer's Disease Neuroimaging Initiative database (“ADNI1 cohort”), the National Institute on Aging Late-Onset Alzheimer's Disease Family Study (referred to herein as the “LOAD study”), and a study by Zhang et al. (referred to herein as the “Cell Study”). It was found that by limiting the SNPs of interest to these regions, the risk of false positives is reduced. The SNP data were obtained from the Alzheimer's Disease Neuroimaging Initiative (ADNI) database. Details of the LOAD study are found in Lee, J. H. et al., “Analyses of the National Institute on Aging Late-Onset Alzheimer's Disease Family Study: Implication of Additional Loci,” 65 Arch. Neurol. 1518-1526 (2008). Details of the Cell Study are found in Zhang, B. et al., “Integrated Systems Approach Identifies Genetic Nodes and Networks in Late-Onset Alzheimer's Disease,” 153 Cell 707-720 (2013).

In addition to examining the regions of interest in all patients, subset-specific analyses were also performed based on both gender and APOE status. Reasoning that different subsets of AD patients may have differences in the biological factors driving their disease, the data was stratified by (a) gender due to the findings above and by (b) APOE genotype as the gene is observed to be most significantly associated with AD. Carriers of the APOE ε4 (APOE4) allele have a significantly increased risk of AD for reasons that are incompletely understood, and several clinical studies have found putative differences in response to therapy based on APOE4 status.

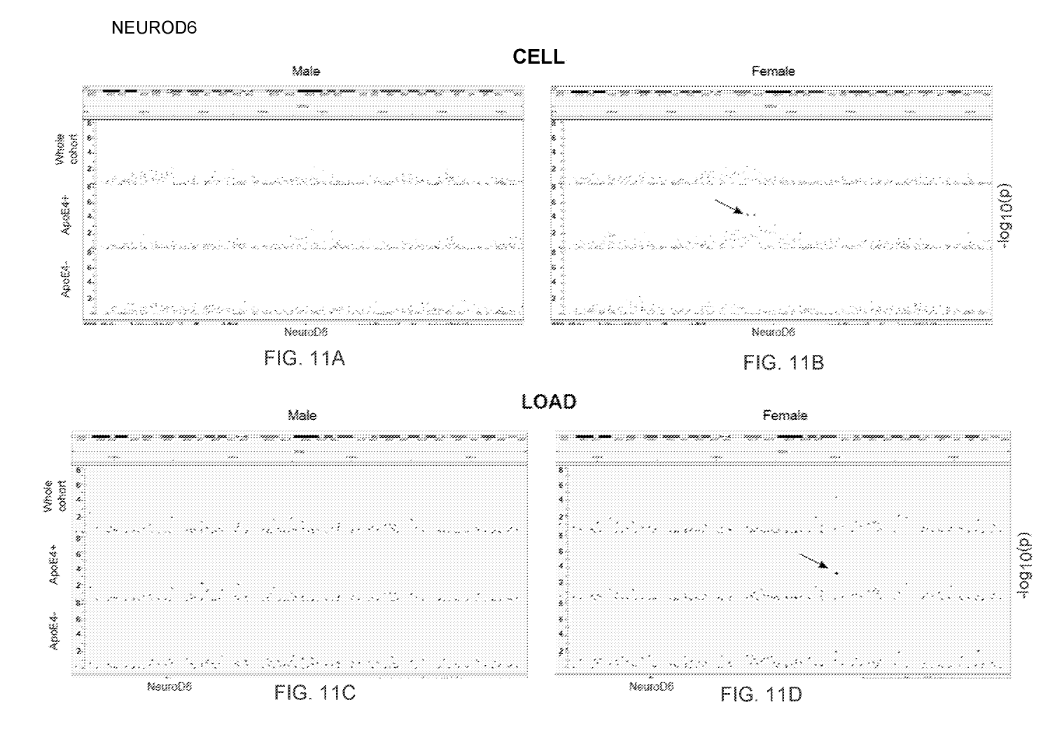

SNPs in the region of NEUROD6 were then associated with AD, specifically in APOE4+ women in both the ADNI1 and LOAD cohorts. These NEUROD6 SNPs are illustrated in FIGS. 11A-11D. The targeted gene association testing from the SNP datasets was conducted using PLINK with patient subsets defined by gender and APOE4 status. Results were visualized using the Integrative Genomics Viewer (IGV). Details of the PLINK application are found in Purcell, S. et al., “PLINK: A Tool Set for Whole-Genome Association and Population-Based Linkage Analyses,” 81 The American Journal of Human Genetics 559-575 (2007). Details of the Integrative Genomics Viewer are found in Robinson, J. T. et al., “Integrative genomics viewer,” 29 Nat. Biotechnol. 24-26 (2011).

FIGS. 11A-11D illustrate SNPs in the region of NEUROD6 that are found to be associated with AD specifically in APOE4+ women in both the ADNI1 and LOAD cohorts.

In FIGS. 11A and 11B, the plot shows a “cone” of disease associated SNPs around NEUROD6 in APOE4+ female patients, but not in APOE4+ male, APOE4− female, or APOE4− male patients. It is observed that the top AD-associated SNPs (related to NEUROD6 in APOE4+ female patients in ADNI) include rs1917011 (p<3.82e-5 in female APOE4+ patients, p<0.692 in male APOE4+, p<0.844 in female APOE4−), rs2159766 (p<3.82e-5 in female APOE4+, p<0.771 in male APOE4+, p<0.624 in female APOE4−), and rs12701070 (p<3.82e-5 in female APOE4+, p<0.561 in male APOE4+, p<0.624 in female APOE4−).

In FIGS. 11C-D, the plots show disease associated SNP near NEUROD6 in APOE4+ female patients, but not in APOE4+ male, APOE4− female, or APOE4− male patients from the LOAD dataset. This SNP includes rs6972352 (p<0.00049 in female APOE4+, p<0.2247 in male APOE4+, and p<0.010 in female APOE4−).

In order to obtain the strongest possible AD-relevant signals, the analysis of the LOAD dataset was restricted to only those patients with an AD diagnosis confirmed by autopsy. Similarly, in the analysis of the Cell Study dataset, controls and patients having a diagnosis of Huntington's disease, for example, were excluded.

In the ADNI1 study, 757 patients were genotyped via Illumina 610 Quad array SNP chip, and 389 patients were categorized as either AD patients or healthy controls. The remaining patients in the ADNI1, for example, those with mild-cognitive impairment, MCI, were excluded from further consideration. GWAS was also run on patient sub-cohorts and stratified by APOE4 status, gender, and a combination of both. The effect sizes for each group were as follows: unstratified=175 AD patients, 214 healthy controls (HC); APOE4−=58 AD patients, 156 HC; APOE4+=117 AD patients, 58 HC; Female APOE4−=31 AD patients, 73 HC; Female APOE4+=51 AD patients, 26 HC; Male APOE4−=27 AD patients, 88 HC; Male APOE4+=66 AD patients, 32 HC; Male all=93 AD patients, 115 HC; Female all=82 AD patients, 99 HC.

In the LOAD dataset, 1985 patients and 2058 controls were genotyped via Illumina Human 610 Quad v1B SNP chip. Patients included in the present analysis were limited, for example, to those who had an AD diagnosis confirmed by autopsy. Unstratified subjects in the present analysis included 440 patients and 2058 controls. The numbers of subjects in the stratified subsets were as follows: APOE4−=99 AD patients, 1256 HC; APOE4+=341 AD patients, 802 HC; Female APOE4−=74 AD patients, 773 HC; Female APOE4+=230 AD patients, 483 HC; Male APOE4−=25 AD patients, 483 HC; Male APOE4+=111 AD patients, 319 HC; Male all=136 AD patients, 802 HC; Female all=304 AD, 1256 HC.

In the Cell Study, 374 patients and 366 controls were genotyped via Illumina HumanHap650Y SNP chip. Subjects with a diagnosis of Huntington's disease were excluded from the analysis. Unstratified subjects included 371 patients and 159 controls. The numbers of subjects in the stratified subsets included: APOE4−=209 AD patients, 130 HC; APOE4+=162 AD patients, 29 HC; Female APOE4−=129 AD patients, 34 HC; Female APOE4+=90 AD patients, 5 HC; Male APOE4−=80 AD patients, 96 HC; Male APOE4+=72 AD patients, 24 HC; Male all=152 AD patients, 120 HC; Female all=219 AD patients, 39 HC. Because patients and controls with a diagnosis of Huntington's disease were removed, smaller numbers of controls were available, especially in the highly stratified subsets. Specifically, the female APOE+ cohort contained only 5 controls, which may be one reason that SNPs significant observed in female APOE4+ patients in the other two datasets were not observed to be significant in this subset.

### Propensity Plot Analysis

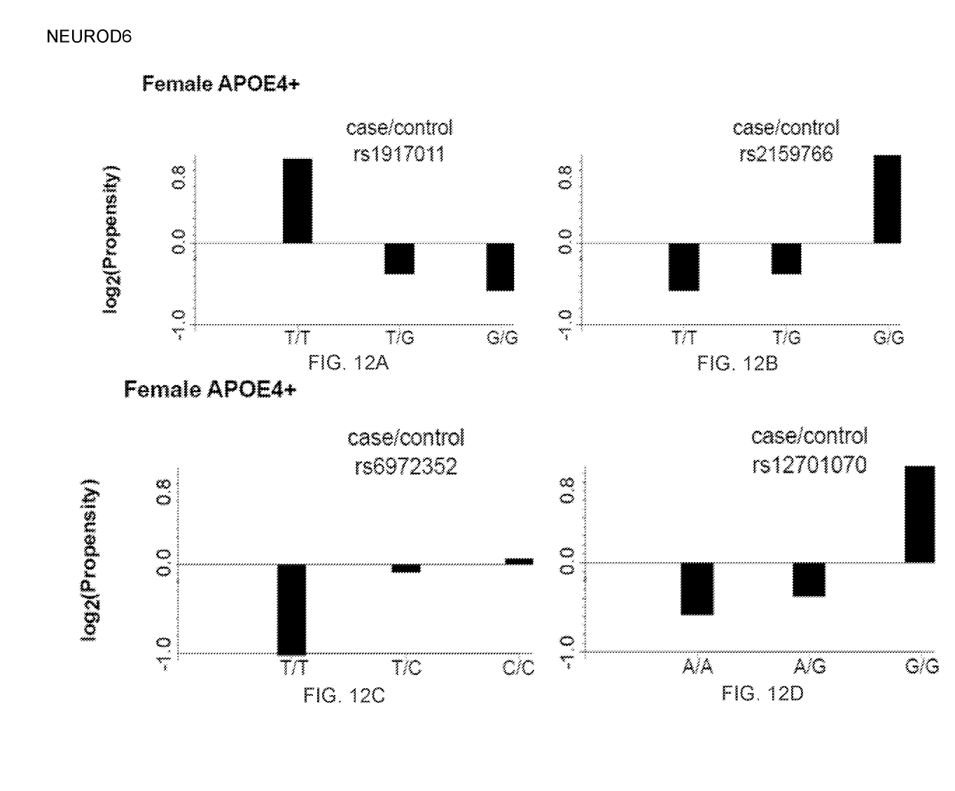

As indicated, it was found that SNPs in the region of NEUROD6 were associated with AD specifically in APOE4+ women in both the ADNI1 and LOAD cohorts. The propensity plotting method of the present disclosure was employed to visualize the specific influence of these SNPs. FIGS. 12A-12D illustrate propensity plots of the specific influence of the NEUROD6 SNPs with disease propensity. As shown in the figure, the positive values indicate a disease risk propensity in APOE4+ female patients, and the negative values indicate a protection propensity. The figures show that the status of NEUROD6 SNPs are highly associated with disease propensity.

The propensity score is a measure of preference of a particular SNP genotype to case versus the control subsets of the dataset. Here, the propensity score was calculated using Equation 3.

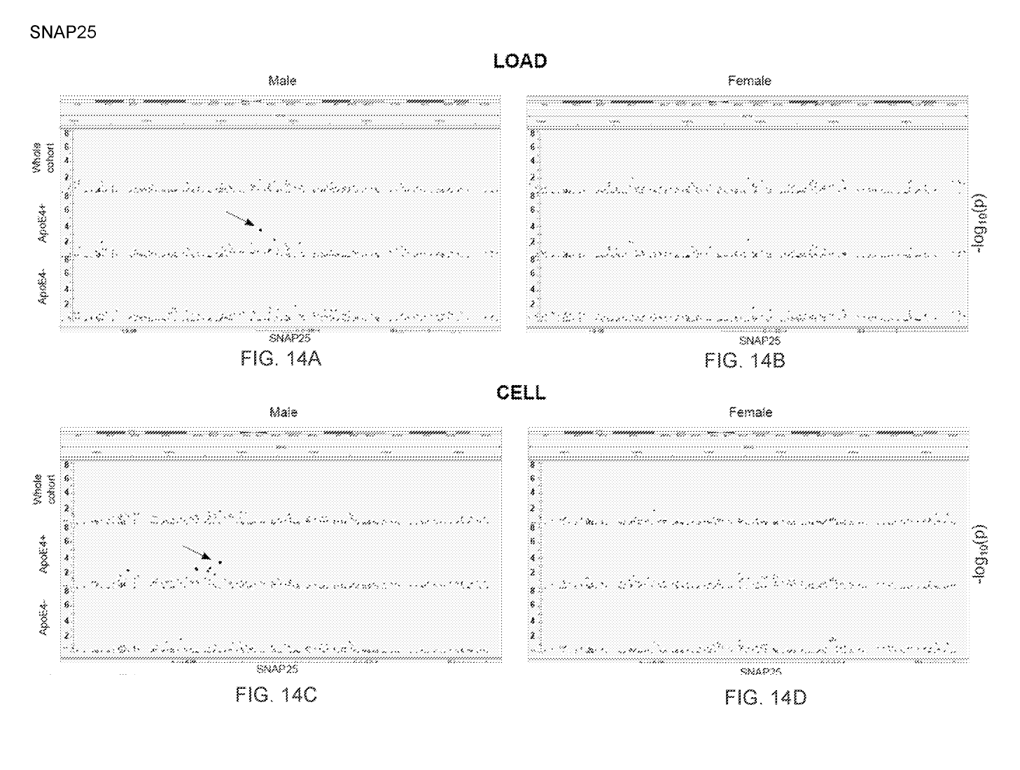

It was also found that SNPs in the region of SNAP25 were associated with AD specifically in APOE4+ men in both the LOAD and Cell datasets. FIGS. 15A-15B illustrate propensity plots for disease risk or disease protection of SNPs in the region of SNAP25 in APOE4+ that are found to be associated with disease propensity in male patients. For each of the top SNAP25 SNPs, the positive values indicate a disease risk propensity in APOE4+ female patients, and the negative values indicate a protection propensity.

### Alzheimer Disease (AD) Study Discussion

Without wishing to be bound by any particular theory, NEUROD6 is a transcription factor involved in neuronal differentiation, and has been shown to increase mitochondrial mass and play a role in response to oxidative stress. This is intriguing because the aging process has a negative impact on mitochondrial function and leads to an increase in mitochondrial DNA mutations, and rates of Alzheimer's increase dramatically with age. APOE also has ties to the mitochondria. The APOE ε4 (APOE4) isoform has been shown to cause mitochondrial damage specifically in neurons. APOE ε4 (APOE4) also has lower antioxidant capability than other isoforms, and amyloid beta induces oxidative stress to a greater extent when APOE ε4 is present. Oxidative stress may also induce hyperphosphorylation of tau, another key factor in AD. Impairment of the transport of mitochondria into axons has been shown to enhance tau phosphorylation and neurodegeneration. It has been shown that oxidative stress induces upregulation of BACE1, an enzyme critical for the production of amyloid beta. It has also been shown that oxidative stress increases production of amyloid precursor protein. Because NEUROD6 confers tolerance to oxidative stress, it has the potential to mitigate some of this damage. Because NEUROD6 expression is lower in women, and APOE4+ individuals have lowered tolerance for ROS damage, it stands to reason that a SNP associated with further impairment of NEUROD6 may put APOE4+ females at particular risk of damage due to oxidative stress. Without wishing to be bound by any particular theory, SNAP25 has a role in synaptic function as part of the SNARE complex, which is involved in synaptic vesicle exocytosis, and has been tied to neurodegeneration. SNARE proteins are sensitive to oxidative stress, with SNAP25 being the most sensitive, which has been proposed to relate mitochondrial dysfunction to reduced synaptic activity in neurodegeneration.

### Identification of Agents for Alzheimer's Therapy

In another aspect, the analysis determined medicines that may restore the expression, for example, of NEUROD6 or SNAP25 downregulated in AD. The methods and systems of the present disclosure were employed to identify specific drugs in the Connectivity Map (CMAP) databases in which the drugs induced significantly higher expression of NEUROD6 or SNAP25 in culture. The CMAP dataset is a large collection of microarray-based transcriptional signatures that includes over 7,000 expression profiles from cultured cells treated with 1,309 compounds. The full CMAP (builds 01 and 02) datasets were obtained from the Broad Institute. A single-sample Wilcox test was used to look for expression profiles from compounds that significantly increased the expression of a gene of interest in the culture after treatment. The output p-values were adjusted for multiple hypothesis testing (FDR p value <0.05).

Table 7 shows unique compounds identified to upregulate or induce enriched expressions of NEUROD6.

TABLE 7
Compounds Identified to Upregulate Enriched NEUROD6 Expression

| CMAP compound name | p.val | adj.p.val |
|---|---|---|
| sodium phenylbutyrate | 8.03E−05 | 4.23E−02 |
| arachidonic acid | 8.22E−05 | 4.23E−02 |
| 2-deoxy-D-glucose | 8.59E−05 | 4.23E−02 |
| fasudil | 8.76E−05 | 4.23E−02 |
| nordihydroguaiaretic acid | 1.04E−04 | 4.23E−02 |
| monastrol | 1.09E−04 | 4.23E−02 |
| tacrolimus | 1.12E−04 | 4.23E−02 |
| quercetin | 1.12E−04 | 4.23E−02 |
| sulindac | 1.14E−04 | 4.23E−02 |
| troglitazone | 1.17E−04 | 4.23E−02 |
| staurosporine | 1.17E−04 | 4.23E−02 |
| troglitazone | 1.22E−04 | 4.23E−02 |
| thalidomide | 1.26E−04 | 4.23E−02 |
| CP-944629 | 1.35E−04 | 4.23E−02 |
| mercaptopurine | 1.40E−04 | 4.23E−02 |
| haloperidol | 1.49E−04 | 4.23E−02 |
| exisulind | 1.57E−04 | 4.23E−02 |
| sirolimus | 1.71E−04 | 4.23E−02 |
| tanespimycin | 1.71E−04 | 4.23E−02 |
| suramin sodium | 1.74E−04 | 4.23E−02 |
| genistein | 1.76E−04 | 4.23E−02 |
| erastin | 1.78E−04 | 4.23E−02 |
| clofibrate | 1.80E−04 | 4.23E−02 |
| LY-294002 | 1.92E−04 | 4.23E−02 |
| tanespimycin | 1.93E−04 | 4.23E−02 |
| LY-294002 | 1.97E−04 | 4.23E−02 |
| prednisolone | 1.99E−04 | 4.23E−02 |
| fulvestrant | 2.01E−04 | 4.23E−02 |
| meteneprost | 2.05E−04 | 4.23E−02 |
| monorden | 2.17E−04 | 4.23E−02 |
| tretinoin | 2.22E−04 | 4.23E−02 |
| nifedipine | 2.30E−04 | 4.23E−02 |
| sulindac sulfide | 2.32E−04 | 4.23E−02 |
| wortmannin | 2.36E−04 | 4.23E−02 |
| MK-886 | 2.46E−04 | 4.29E−02 |
| PF-01378883-00 | 2.59E−04 | 4.38E−02 |
| monorden | 2.82E−04 | 4.65E−02 |
| iloprost | 3.06E−04 | 4.91E−02 |

Details of the Connectivity Map databases are found in Webster, J. A. et al., “Genetic Control of Human Brain Transcript Expression in Alzheimer Disease,” 84 The American Journal of Human Genetics 445-458 (2009). Table 8 shows unique compounds identified to upregulate or induce enriched expressions of SNAP25.

TABLE 8
Compounds Identified to Upregulate Enriched SNAP25 Expression

| CMAP compound name | p.val | adj.p.val |
|---|---|---|
| valproic acid | 2.20E−05 | 1.91E−02 |
| guanabenz | 9.14E−05 | 3.81E−02 |
| karakoline | 8.89E−05 | 3.81E−02 |
| tetracycline | 1.03E−04 | 4.01E−02 |
| diloxanide | 1.28E−04 | 4.45E−02 |
| metoprolol | 1.38E−04 | 4.52E−02 |
| yohimbic acid | 1.59E−04 | 4.75E−02 |
| azapropazone | 1.63E−04 | 4.75E−02 |
| proguanil | 1.93E−04 | 4.92E−02 |

Several of these compounds show promise in lab experiments, for example, in mouse models of AD. Sodium phenylbutyrate, for example, has been proposed as a therapeutic for neurodegenerative diseases due to its ability to increase neurotrophic factors in brain cells, along with the fact that it is safe, orally delivered, and crosses the blood brain barrier. 2-Deoxy-D-Glucose, for example, has been shown to reduce pathology in a female mouse model of AD.

As indicated, NEUROD6 is observed in the present analysis to be most significant in the female population. Without wishing to be bound by any particular theory, estrogen signaling appears to stimulate the production of enzymes, such as glutathione peroxidase, that protect the mitochondria against oxidative stress. Thus, the loss of estrogen upon age may leave women more susceptible to mitochondrial damage associated with impairment of NEUROD6 production and the resultant loss of protective effects. Genistein, for example, is found to be significantly elevate expression of NEUROD6 among the list of compounds from Table 7. Genistein also has been proposed as a means to replace the protective effect of estrogen on mitochondria in aging women.

Valproic acid, for example, is observed to significantly elevate SNAP25 expression and is known to have neuroprotective properties. In studies in mouse models of AD, valproic acid was demonstrated to protect against loss of neurons and limit Aβ production and behavioral deficits. Karakoline, for example, is a nicotinic receptor agonist that has been shown to improve cognitive function in a mouse model of AD. Tetracycline, for example, has been shown to protect from Aβ toxicity in C elegans, and its derivatives are actively being explored as potential therapeutics in mouse models of AD. The analysis suggests that these compounds may be employed for the treatment of AD, particularly in APOE4+ men.

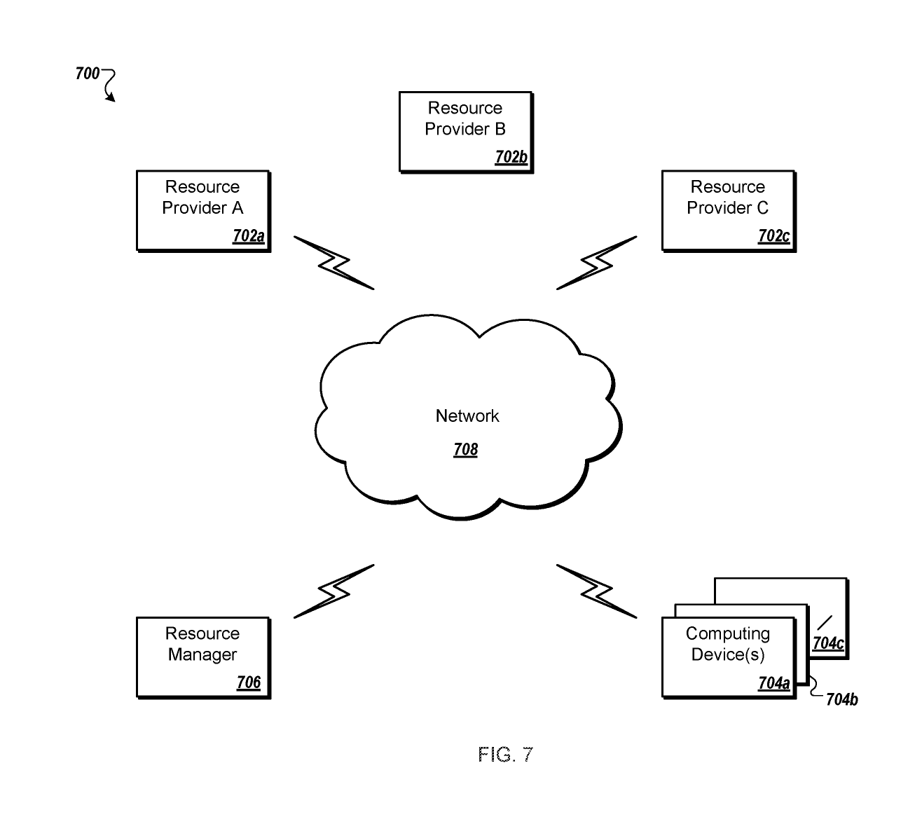

As shown in FIG. 7, an implementation of an exemplary cloud computing environment 700 for automated review of genomic data to identify downregulated and/or upregulated gene expression indicative of a disease or condition is provided. The cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

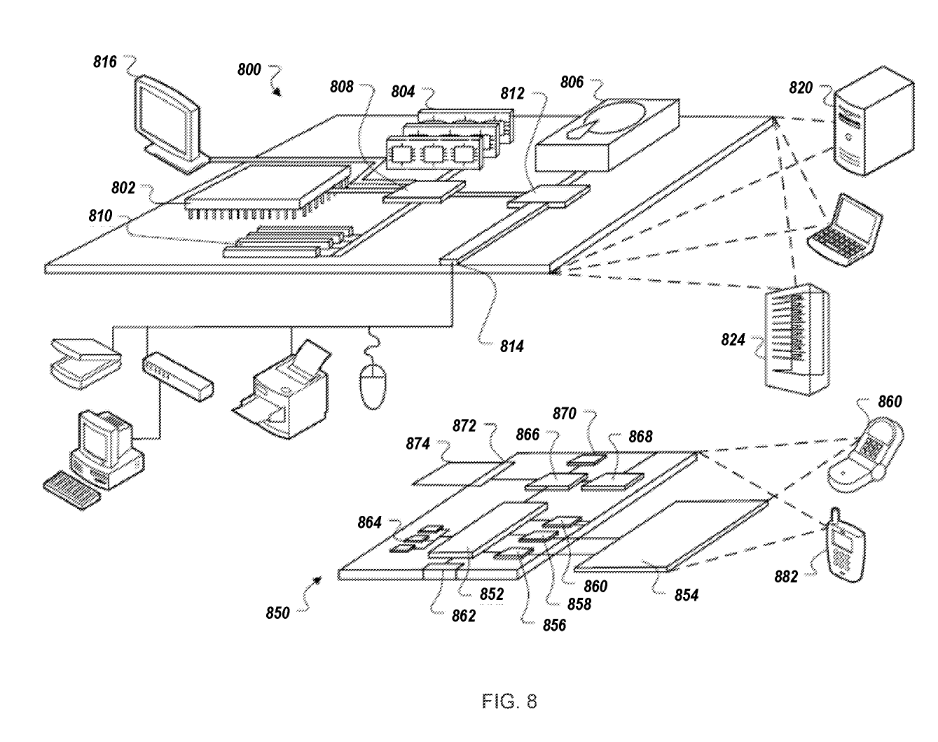

FIG. 8 shows an example of a computing device 800 and a mobile computing device 850 that can be used to implement the techniques described in this disclosure. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, tablet computers, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer- or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 852), perform one or more methods, such as those described above. The instructions can also be stored by one or more storage devices, such as one or more computer- or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth®, Wi-Fi™, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In view of the structure, functions and apparatus of the systems and methods described here, in some implementations, a system and method for automated review of genomic data to identify downregulated and/or upregulated gene expression indicative of a disease or condition are provided. Having described certain implementations of methods and apparatus for supporting automated review of genomic data to identify downregulated and/or upregulated gene expression indicative of a disease or condition, it will now become apparent to one of skill in the art that other implementations incorporating the concepts of the disclosure may be used. Therefore, the disclosure should not be limited to certain implementations, but rather should be limited only by the spirit and scope of the following claims.

Methods described herein enable for the first time the integration of heterogeneous data sets (e.g., data collected from multiple sources and/or selected or filtered for multiple assays or markers) and provides useful information regarding categories of patient populations that require or likely to respond to particular therapies. Accordingly, the present invention provides methods for treating a subset of a patient population having a set of defined genetic profiles (e.g., gene features). In particular, methods described herein are useful for treating a disease characterized by a broad range of heterogenicity observed in affected individuals (i.e., patient populations).

Based on the information obtained in accordance with the present disclosure, it is possible to design and monitor therapeutic regimens that are suitable for a particular population of patients with one or more genetic features, so as to optimize effectiveness of the therapy.

As described herein, in some embodiments, categories of populations are divided by subsets of individuals having differential gene expressions of certain disease marker or markers. Individuals include patients, healthy or normal individuals, as well as those at risk of developing a disease or a condition. Patients include those who have been diagnosed with a disease or a condition and those who have a disease or a condition but have not been diagnosed. In some embodiments, a disease or a condition manifests itself as symptomatic or asymptomatic.

In some embodiments, such subsets involve differential “status” (or genotype) of a marker (i.e., a marker gene). More, specifically, one subset represents a population of individuals having a “positive (+)” genotype, while a second subset represents a population of individuals having a “negative (−)” genotype. An individual with a positive genotype is generally referred to as a carrier of a particular allele. An individual with a negative genotype is generally referred to as a non-carrier of a particular allele. As described in further detail below, in case of AD, non-limiting examples of marker genes include APOE4.

In some embodiments, such subsets involve categorizing by the gender of individuals in a population. In some embodiments, a disease or a condition of interest exhibits gender-dependent features, such as differential pathogenesis, including differences in the onset, severity, duration, survival, and/or symptoms of a disease or condition. In some embodiments, subsets of individuals show differential responsiveness to a particular therapy, including types of drugs, effective dosage and other therapeutic regimens, side effects, and so on. In some embodiments, subsets of individuals show differential responsiveness to different combinations of drugs (e.g., combination therapy).

As used herein, differential responsiveness refers to statistically significant variations observable within a population of individuals in response to a particular therapy.

Accordingly, the present invention provides methods for treating a subset of a patient population having a set of defined genetic profiles (e.g., gene features). In particular, methods described herein are useful for treating a disease characterized by a broad range of heterogenicity observed in affected individuals (i.e., patient populations). In some embodiments, methods described herein are useful for treating Alzheimer's disease (AD).

Additionally, other markers include genes whose expression levels vary significantly when a sample from a diseased or affected tissue or tissues is compared to a control or reference from a healthy tissue. Variations or differences in expression levels may refer to those of a gene or gene product, a form of a gene or gene product (e.g., methylation state of a gene; capping or spliced condition of an RNA gene product, phosphorylation state of a protein gene product, etc.). In some embodiments, an activity level with respect to at least one biological function or type of a gene or gene product is significantly increased or decreased in a sample from a diseased or affected tissue or tissues, as is compared to a control or reference from a healthy tissue. As described herein, in some embodiments, such variations or differences in expression levels and/or activity levels may be correlated with an associated genetic marker (e.g., a single nucleotide polymorphism (“SNP”) or other sequence variation, copy number variation, heterogeneity, etc.), wherein the genetic feature is associated or correlated with a particular disease, disorder, condition, state, or symptom or phenotype thereof. As such, determination or detection of such SNPs provides a means for an indirect readout by correlation (e.g., proxy) that is indicative of the variations or differences in expression levels and/or activity levels in certain tissue or tissues of interest. In certain embodiments, this is particularly useful because of difficulty or inaccessibility in obtaining certain tissues for measuring a tissue-specific expression and/or activity of a marker gene or gene product. These include, without limitation, nervous tissues/cells (such as spinal cord and brain tissues) and embryonic or fetal tissues in utero.

Accordingly, genetic markers that provide genotypic information are useful for carrying out the methods described herein. The art is familiar with techniques used to determine such genetic markers by genotyping. These markers include alleles that are either present or absent (i.e., positive or negative) such that an individual is either a carrier or non-carrier of the gene.

Genotyping is the process of determining differences in the genetic make-up (genotype) of an individual by examining the individual's DNA sequence using biological assays and comparing it to another individual's sequence or a reference sequence. Current methods of genotyping typically include restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads.

In addition, SNPs associated with one or more disease-related genes may also be determined by genotyping. A single-nucleotide polymorphism is a DNA sequence variation occurring when a single nucleotide—A, T, C or G—in the genome (or other shared sequence) differs between members of a biological species or paired chromosomes in a human. Most of the common SNPs identified to date have two alleles. The genomic distribution of SNPs is not homogenous; SNPs usually occur in non-coding regions more frequently than in coding regions or, in general, where natural selection is acting and fixating the allele of the SNP that constitutes the most favorable genetic adaptation.

Within a population, SNPs can be assigned a minor allele frequency—the lowest allele frequency at a locus that is observed in a particular population. This is simply the lesser of the two allele frequencies for single-nucleotide polymorphisms. There are variations between human populations, so a SNP allele that is common in one geographical or ethnic group may be much rarer in another.

These genetic variations may underlie differences in susceptibility to certain diseases. In some situations, the severity of illness and the way a body responds to treatments are also manifestations of genetic variations. For example, a single base mutation in the APOE (apolipoprotein E) gene is associated with a higher risk for Alzheimer's disease. Variations in the DNA sequences of humans can also affect how humans develop diseases and respond to pathogens, chemicals, drugs, vaccines, and other agents. Accordingly, SNPs can be useful for personalized medicine, as described in the present disclosure.

The present disclosure puts the use of SNPs in practice for genome-wide association studies (GWAS), e.g. as high-resolution markers in gene mapping related to diseases or normal traits. The knowledge of SNPs will help in understanding pharmacokinetics (PK) or pharmacodynamics, e.g., how drugs act in individuals with different genetic variants. A wide range of human diseases like cancer, infectious diseases (AIDS, leprosy, hepatitis, etc.), autoimmune, neuropsychiatric, Sickle-cell anemia, β Thalassemia and Cystic fibrosis might result from SNPs. Diseases with different SNPs may become relevant pharmacogenomic targets for drug therapy. Some SNPs are associated with the metabolism of different drugs. SNPs without an observable impact on the phenotype are still useful as genetic markers in genome-wide association studies, because of their quantity and the stable inheritance over generations.

Analytical methods to discover novel SNPs and detect known SNPs include but are not limited to: DNA sequencing; capillary electrophoresis; mass spectrometry; single-strand conformation polymorphism (SSCP); electrochemical analysis; denaturating HPLC and gel electrophoresis; restriction fragment length polymorphism; and hybridization analysis. Useful tools for SNPs analysis include but are not limited to: GWAsimulator; PLINK (module); Affymetrix; International HapMap Project; SNP array; Short tandem repeat (STR); Single-base extension; Snpstr; Tag SNP; TaqMan; and Variome.

Determination of such genetic markers, optionally in combination, provides meaningful information for designing, establishing, monitoring and/or altering a course of treatment for an associated disease or disorder. A particular subset or subsets of a patient population may be more or less responsive to a certain drug or therapy, depending on genetic profiles which can be determined by methods described herein.

In case of AD, for example, the APOE4 status (i.e., APOE4 carriers vs. non-carriers) is one factor to take into account for determining suitable therapeutic regimens. In some embodiments, APOE4 carriers are more likely to respond to certain AD therapies, as compared to non-carriers, or vice versa. Information that can be obtained in accordance with the present disclosure may be used to outline or anticipate suitable therapeutic regimens that are more likely to be effective for a particular subset of patients. Thus, in some cases, a therapy may be initiated accordingly, or an existing therapy may be ceased or modified accordingly.

To date, standard AD therapies include but are not limited to: cholinesterase inhibitors, such as Donepezil (Aricept); Rivastigmine (Exelon) and galantamine (Razadyne); glutamate regulators, such as Memantine (Namenda); Antidepressants, such as citalopram (Celexa), fluoxetine (Prozac), paroxetine (Paxil), and sertraline (Zoloft); Anxiolytics, such as lorazepam (Ativan) and oxazepam (Serax); Antipsychotic medications, such as aripiprazole (Abilify), haloperidol (Haldol), and olanzapine (Zyprexa); Vitamin E; Hormone replacement therapy (HRT), such as estrogen; Sensory therapies, such as music therapy and art therapy; and alternative therapies, including coenzyme Q10, coral calcium, huperzine A, and omega-3 fatty acids.

What is claimed is:

1. A system for identifying one or more genes that are downregulated due to a disease or condition, the system comprising:
a processor; and
a memory having instructions stored thereon, wherein the instructions, when executed by the processor, cause the processor to:
(a) access genomic data of a first cohort of individuals, wherein the first cohort comprises a group of individuals having the disease or condition and a control group of individuals that do not have the disease or condition;
(b) identify, from the genomic data of at least a subset of the first cohort, a set of one or more genes each of which is differentially expressed by individuals in the group having the disease or condition compared with the control group;
(c) access single-nucleotide polymorphism (SNP) data of a second cohort of individuals different from the first cohort;
(d) identify, from the SNP data of at least a subset of the second cohort, a plurality of SNPs associated with the disease or condition; and
(e) determine an intersection between the set of one or more genes identified in (b) and the plurality of SNPs associated with the disease or condition identified in (d) to identify one or more genes that are downregulated due to the disease or condition.

2. The system of claim 1, wherein the instructions, when executed by the processor, further cause the processor to:
(f) access a drug database; and
(g) identify one or more drug candidates for restoring expression of at least one of the one or more downregulated genes identified in (e).

3. The system of claim 1 or 2, wherein the disease or condition is Alzheimer's disease (AD).

4. A system for visualizing location and/or significance of a set of identified single-nucleotide polymorphisms (SNPs) in relation to one or more identified gene via propensity plotting, the system comprising:
a processor; and
a memory having instructions stored thereon, wherein the instructions, when executed by the processor, cause the processor to:
determine, for each of a plurality of SNPs identified in a Genome-Wide Association Study (GWAS) of a dataset, a propensity score for each of one or more allelic states of the SNP, wherein the propensity score for a given allelic state is a measure of prevalence of the allelic state of the SNP in a case subset versus a control subset of the dataset, where the case subset corresponds to subjects with a given disease or condition and the control subset corresponds to subjects who do not have the disease or condition;
display, for each of the plurality of SNPs identified in the GWAS of the dataset, a graphical representation of the propensity score for each of the one or more allelic state(s) of the SNP, thereby enabling a user to distinguish allelic states having strong association with either the case subset or the control subset of the dataset.

5. The system of claim 4, wherein the graphical representation comprises an x-y plot, with each of a plurality of allelic states of a given SNP represented by a discrete location along either the x or y axis, and a value of the propensity score represented graphically along the other axis.

6. A system for performing a search of one or more large datasets containing gene expression data, at least a portion of which is not normalized, to identify samples in the one or more large datasets having an input gene set that is significantly upregulated only, downregulated only, or either up OR downregulated, the system comprising:
a processor; and
a memory having instructions stored thereon, wherein the instructions, when executed by the processor, cause the processor to:
determine a normalized enrichment score for each of a plurality of samples in the one or more large datasets, wherein the normalized enrichment score for a given sample is a measure of whether a given input gene set comprising a plurality of genes is upregulated, downregulated, or both in the given sample;
convert the normalized enrichment score for a plurality of samples to z-scores having a standard Gaussian distribution; and
identify a subset of the plurality of samples in the one or more large datasets in which the given input gene set is upregulated, downregulated, or both.

7. The system of claim 6, wherein the normalized enrichment score for a given sample comprises one or more of:
(i) a measure of significance of differential expression of probes annotated to a gene of interest against all other probes in the given sample;
(ii) a signal-to-noise ratio associated with the input gene set in the given sample compared to other genes in the given sample; and
(iii) a difference between the number of genes in the given sample and the number of genes in the input gene set.

8. The system of claim 6 or 7, wherein the instructions, when executed by the processor, cause the processor to identify conditions and/or treatments that upregulate or downregulate a given pathway.

9. The system of any one of claims 6, 7, and 8, wherein the instructions, when executed by the processor, cause the processor to identify one or more other conditions and/or diseases whose expression profiles are similar to that of a disease of interest.

10. The system of claim 9, wherein the instructions, when executed by the processor, cause the processor to use the identified one or more other conditions and/or diseases whose expression profiles are similar to that of the disease of interest to identify one or more pathways common between the identified one or more other conditions and/or diseases and the disease of interest.

11. The system of claim 9 or 10, wherein the instructions, when executed by the processor, cause the processor to use the identified one or more other conditions and/or diseases whose expression profiles are similar to that of the disease of interest to identify one or more known treatments for the one or more other conditions and/or diseases.

12. A method for identifying one or more genes that are downregulated due to a disease or condition, the method comprising:
(a) identifying, by a processor of a computing device, a set of one or more genes each of which is differentially expressed by individuals in a group having the disease or condition compared with individuals in a control group that do not have the disease or condition, said identifying based on data corresponding to a first cohort of individuals;
(b) accessing, by the processor, single-nucleotide polymorphism (SNP) data of a second cohort of individuals different from the first cohort and identifying, by the processor, a plurality of SNPs associated with the disease or condition; and
(c) determining, by the processor, an intersection between the set of one or more genes identified in step (a) and the plurality of SNPs associated with the disease or condition identified in step (b) to identify one or more genes that are downregulated due to the disease or condition.

13. The method of claim 12, further comprising:
(d) accessing, by the processor, a drug database and identifying, by the processor, one or more drug candidates for restoring expression of at least one of the one or more downregulated genes.

14. The method of claim 12 or 13, wherein the one or more downregulated genes identified in step (c) is/are indicative of an upstream signal rather than a downstream signal resulting from disease pathology.

15. A method for performing a search of one or more large datasets containing gene expression data, at least a portion of which is not normalized, to identify samples in the one or more large datasets having an input gene set that is significantly upregulated only, downregulated only, or either up OR downregulated, the method comprising:
determining, by a processor of a computer, a normalized enrichment score for each of a plurality of samples in the one or more large datasets, wherein the normalized enrichment score for a given sample is a measure of whether a given input gene set comprising a plurality of genes is upregulated, downregulated, or both in the given sample;
converting, by the processor, the normalized enrichment score for a plurality of samples to z-scores having a standard Gaussian distribution; and
identifying, by the processor, a subset of the plurality of samples in the one or more large datasets in which the given input gene set that is upregulated, downregulated, or both.

16. The method of claim 15, wherein the normalized enrichment score for a given sample comprises one or more of:
(i) a measure of significance of differential expression of probes annotated to a gene of interest against all other probes in the sample;
(ii) a signal-to-noise ratio associated with the input genes in the sample compared to other genes in the sample; and
(iii) a difference between the number of genes in the sample and the number of genes in the input gene set.

17. The method of claim 15 or 16, comprising identifying, by the processor, conditions and/or treatments that upregulate or downregulate a given pathway.

18. The method of any one of claims 15, 16, and 17, comprising identifying, by the processor, one or more other conditions and/or diseases whose expression profiles are similar to that of a disease of interest.

19. The method of claim 18, comprising using the identified one or more other conditions and/or diseases whose expression profiles are similar to that of the disease of interest to identify one or more pathways common between the identified one or more other conditions and/or diseases and the disease of interest.

20. The method of claim 18 or 19, comprising using the identified one or more other conditions and/or diseases whose expression profiles are similar to that of the disease of interest to identify one or more known treatments for the one or more other conditions and/or diseases.

21. A method comprising steps of:
determining one or more of gender and ApoE4 status for a subject; and
detecting in one or more samples from the subject a genetic feature selected from the group consisting of:
a genetic feature indicative of NEUROD6 expression, activity, or combination thereof in the subject's brain as compared with an appropriate reference;
a genetic feature indicative of SNAP25 expression, activity, or combination thereof in the subject as compared with an appropriate reference; and
combinations thereof.

22. The method of claim 21, further comprising a step of administering Alzheimer's therapy, including one or more agents, to the subject if the subject is either:
i) ApoE4+ female and has a NEUROD6 feature indicating a level, expression, activity, or function of NEUROD6 in the subject's brain that is significantly lower than that of a normal NEUROD6 reference; or
ii) ApoE4+ male and has a SNAP25 feature indicating a level, expression, activity, or function of SNAP25 expression in the subject's brain that is significantly lower than that of a normal SNAP25 reference.

23. The method of claim 22, wherein the step of administering comprises administering an agent whose administration correlates with increased NEUROD6 brain level, expression, function, or activity.

24. The method of claim 22, wherein the agent is selected from the following:
sodium phenylbutyrate, arachidonic acid, 2-deoxy-D-glucose, fasudil, nordihydroguaiaretic acid, monastrol, tacrolimus, quercetin, sulindac, troglitazone, staurosporine, troglitazone, thalidomide, CP-944629, mercaptopurine, haloperidol, exisulind, sirolimus, tanespimycin, suramin sodium, genistein, erastin, clofibrate, LY-294002, tanespimycin, LY-294002, prednisolone, fulvestrant, meteneprost, monorden, tretinoin, nifedipine, sulindac, ulfide, wortmannin, MK-886, PF-01378883-00, monorden, iloprost, and combinations thereof.

25. The method of claim 22, wherein the step of administering comprises administering an agent whose administration correlates with increased SNAP25 brain level, expression, function, or activity.

26. The method of claim 25, wherein the agent is selected from the following:
valproic acid, guanabenz, karakoline, tetracycline, diloxanide, metoprolol, yohimbic acid, azapropazone, proguanil, and combinations thereof.

27. The method of claim 22, wherein the agent is or comprises a cholinesterase inhibitor.

28. The method of claim 27, wherein the agent is or comprises donepezil, rivastigmine, or galantamine.

29. The method of claim 22, wherein the agent is or comprises a glutamate regulator.

30. The method of claim 29, wherein the agent is or comprises memantine.

31. The method of claim 22, wherein the agent is or comprises an antidepressant, an anxiolytic, or an antipsychotic.

32. The method of claim 31, wherein:
the antidepressant is selected from the group consisting of citalopram, fluoxetine, paroxetine, sertraline, and combinations thereof;
the anxiolytic is selected from the group consisting of lorazepam, oxazepam, and combinations thereof; and
the antipsychotic is selected from the group consisting of ariprazole, baloperidol, olanzapine, and combinations thereof.

33. The method of claim 22, wherein the agent is or comprises a beta secretase inhibitor, a gamma secretase inhibitor, or combinations thereof.

34. The method of claim 22, wherein the agent is or comprises an antibody agent that binds specifically to amyloid beta or tau.

35. The method of claim 34, wherein the antibody agent is an intact antibody, an antigen-binding fragment thereof, or combination thereof.

36. The method of claim 21 or claim 22 wherein the NEUROD6 feature is or comprises a SNP.

37. The method of claim 21 or claim 22 wherein the SNAP25 feature is or comprises a SNP.

38. The method of claim 37, wherein the step of detecting a genetic feature comprises:
obtaining a sample from the subject; and
processing the sample by contacting it with reagents sufficient to hybridize with or amplify the SNP.

39. The method of claim 21 or 22, wherein the NEUROD6 reference is or comprises a NEUROD6 brain level, expression, function, or activity in normal females.

40. The method of claim 21 or 22, wherein the NEUROD6 reference or the SNAP25 reference is a level or range or expression, function, or activity observed in a population of normal individuals not suffering from or being treated for Alzheimer's Disease.

41. The method of any one of claims 21, 22, and 40, wherein the NEUROD6 reference or the SNAP25 reference is a historical reference.

42. The method of any one of claims 21, 22, and 40, wherein the NEUROD6 reference or the SNAP25 reference is a reference level, expression, function, or activity determined in a sample from the subject at an earlier time.

43. The method of claim 21, wherein the step of determining ApoE4 status in a subject comprises:
obtaining a sample from the subject; and
processing the sample by contacting it with reagents sufficient to hybridize with or amplify ApoE4 nucleic acids in the sample, or to bind to or react with ApoE4 protein.

44. The method of claim 21, wherein the step of detecting a genetic feature comprises:
obtaining a sample from the subject; and
processing the sample by contacting it with reagents sufficient to hybridize with or amplify NEUROD6 nucleic acids in the sample, or to bind to or react with NEUROD6 protein such that the subject's brain level of NEUROD6 is determined.

45. The method of claim 44, wherein the sample does not comprise brain tissue, and the subject's brain level of NEUROD6 is determined by proxy.

46. The method of claim 21, wherein the step of detecting a genetic feature comprises:
obtaining a sample from the subject; and
processing the sample by contacting it with reagents sufficient to hybridize with or amplify SNAP25 nucleic acids in the sample, or to bind to or react with SNAP25 protein.

47. The method of claim 46, wherein the sample does not comprise brain tissue, and the subject's brain level of SNAP25 is determined by proxy.

48. The method of claim 47, wherein the step of detecting a genetic feature comprises:
obtaining a sample from the subject; and
processing the sample by contacting it with reagents sufficient to hybridize with or amplify the SNP.
